(19) United States
(12) Patent Application Publication
(10) Pub. No.: US 2003/0177547 A1
(43) Pub. Date: Sep. 18, 2003

(54) Imprinting in plants to control gene expression

(75) Inventors: Olga Danilevskaya, Johnston, IA (US); Pedro Hermon, Johnston, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc., Johnston, IA

(21) Appl. No.: 10/387,894

(22) Filed: Mar. 13, 2003

Related U.S. Application Data
(60) Provisional application No. 60/363,861, filed on Mar. 13, 2002.

(51) Int. Cl. C12Q 1/68; A01H 5/00

(52) U.S. Cl. 800/320.1; 435/6

(57) ABSTRACT

Compositions and methods for identifying imprinting and genes regulated by imprinting are provided. The methods involve an analysis of the nucleotide sequence and the identification of CpG islands. At least two islands are involved in imprinting. Thus, genes can be identified that are differentially expressed based on parental inheritance. In this manner, the methods are useful for determining the propensity of a gene to be influenced by imprinting. Such analysis involves determining the pattern of imprinting for cells of interest.

It is further recognized that DNA constructs can be constructed which show differential expression depending upon the parent-of-origin. To silence a paternally inherited allele, at least two CpG islands are utilized in the construct.

This application claims the benefit of, and hereby incorporates by reference, U.S. Provisional Patent Application No. 60/363,861, filed Mar. 13, 2002.

### BACKGROUND OF THE INVENTION

Genomic imprinting is an epigenetic modification of a specific parental chromosome in the gamete or zygote that leads to monoallelic or differential expression of the two alleles of a gene in somatic cells of the offspring. The general assumption is that maternally- and paternally-transmitted genes are expressed at equivalent levels in progeny. However, non-equivalent expression of the maternally- and paternally-transmitted genes was described in 1970 and 1983 in plants (maize) and mammals, respectively (Alleman M, Plant Mol Biol. (2000) 43:147-61). This phenomenon, named imprinting, is defined as epigenetic gene silencing that is set in the male or female germ lines, resulting in a differential expression of maternally- and paternally-derived alleles. Imprinting affects various essential cellular and developmental processes, including intercellular signaling, RNA processing, cell cycle control, and promotion or inhibition of cellular division and growth.

Many mammalian genes influenced by imprinting have been identified. The first deduction of imprinting at the single gene level involved a transgenic C-myc gene that showed dependence of its expression on paternal inheritance. The silent maternally inherited copy was methylated (Swain et al. (1987) Cell 50:719-727).

The increased attention to imprinting in mammals is due to the recognition of its importance during development and its role in causing several human genetic diseases. Abnormalities of a single gene can affect imprinting of a proximate genomic region and disrupt multiple disease-causing genes, the phenotype depending upon the parental origin of the mutated gene. Imprinted loci have been implicated in disease. For example, disrupted imprinting of a locus is one of the causes of Prader-Willi syndrome (PWS) and Angelman syndrome (AS), which involve mental retardation. PWS also causes obesity, and AS involves gross motor disturbances. Each disorder can be caused by parental-origin specific uniparental disomy (Nicholls et al. (1989) Nature 342:281-285; Knoll et al. (1990) Am. J. Hum. Genet. 47:149-155) or chromosomal deletions (Knoll et al. (1989) Am. J. Hum. Genet. 47:149-155; Mattei et al. (1984) Hum. Genet. 66:313-334).

Genomic imprinting has been implicated in cancer. The work has demonstrated that a balance of maternal and paternal chromosomes is required. A relative imbalance leads to neoplastic growth, and the type of neoplasm depends upon whether there is a maternal or paternal genetic excess. Tumors associated with imprinting include the two embryonic tumors, hydatidiform mole and complete ovarian teratoma, familial paraganglioma or glomus tumor, hepatoblastoma (Rainier et al. (1995) Cancer Res. 55:1836-1838); (Li et al. (1995) Oncogene 11:221-229), rhabdomyosarcoma (Zhan et al. (1994) J. Clin. Invest. 94:445-448), and Ewing's sarcoma (Zhan et al. (1995a) Oncogene 11:2503-2507). Loss of Imprinting (LOI) of IGF2 and H19 have also now been found in many adult tumors, including uterine (Vu et al. (1995) J. Clin. Endocrinol. Metab. 80:1670-1676, cervical (Doucrasy et al. (1996) Oncogene 12:423-430), esophageal (Hibi et al. (1996) Cancer Res. 56:480-482), prostate (Jarrard et al. (1995) Clin. Cancer Res. 1:1471-1478), lung cancer (Kondo et al. (1995) Oncogene 10:1193-1198), choriocarcinoma (Hashimoto et al. (1995) Nat Genet. 9:109-110), germ cell tumors (Van Gurp et al. (1994) J. Natl. Cancer Inst. 86:1070-1075), B W S (Steenman et al. (1994) Nature Genet. 7:433-439); Weksberg et al. (1993) Nature Genet. 5:143-150), and Wilms tumor (Ogawa et al. (1993) Nature Genet 5:408-412). In the case of familial paraganglioma, the transmitting parent is the father (Van der Mey et al. (1989) Lancet 2:1291-1294). The gene has recently been localized to 11q22.3-q23 (Heutink et al. (1994) Eur. J. Hum. Genet 2:148-158).

In angiosperm plants, imprinting is postulated to be essential for endosperm development. In Arabidopsis, the MEA gene regulates cell proliferation by exerting a gametophytic maternal control during seed development. Seeds derived from embryo sacs carrying a mutant mea-1 allele abort after delayed morphogenesis with excessive cell proliferation in the embryo and reduced free nuclear divisions in the endosperm. The mutant mea seeds are able, at a low frequency, to initiate endosperm development, seed coat differentiation, and fruit maturation in the absence of fertilization. See, Vielle-Calzada et al. (1999) Genes & Development 13:2971-2982. The mea mutation affects an imprinted gene expressed maternally in cells of the female gametophyte and after fertilization only from maternally inherited MEA alleles. Paternally inherited MEA alleles are transcriptionally silent in both the young embryo and endosperm.

A consequence of imprinting is the requirement of a 2:1 ratio of maternal to paternal genomes in the endosperm (Haig and Westoby 1991, Am. Nat. 134:147-155). Thus imprinting plays a significant role in the proper development of seed in cereal crops.

Abnormal imprinting has been studied in plants by analysis of gene expression. Methods are needed in the art to identify imprinted genes in plants, to identify genes involved in endosperm development, and to manipulate gene sequences to affect imprinting.

### BRIEF SUMMARY OF THE INVENTION

Compositions and methods for identifying imprinting and genes regulated by imprinting are provided. The methods involve an analysis of the nucleotide sequence and the identification of CpG islands. At least two islands are involved in imprinting. Thus, genes can be identified that are differentially expressed based on parental inheritance. In this manner, the methods are useful for determining the propensity of a gene to be influenced by imprinting. Such analysis involves determining the pattern of imprinting for cells of interest.

It is further recognized that DNA constructs can be created which show differential expression depending upon the parent of origin. To silence a paternally inherited allele, at least two CpG islands are utilized in the construct.

### BRIEF DESCRIPTION OF THE DRAWINGS

FIG. 1 shows the massively parallel signature sequencing (MPSS) analysis of ZmFIE1 expression in embryo and endosperm. The graph represents a distribution of the 17-mer tags (GATCTAGTGTGTGGCTG) in the endosperm and embryo mRNAs generated by MPSS. The recognition site of the restriction enzyme Dpnll, used to generate tags, is GATC. A tag sequence is derived from the ZmFie1 EST (Accession No. AY061964) positioned 112 nt upstream from the polyA tail. The vertical axis represents the frequency of the tags as particles per million (PPM) molecules sequenced on the microbeads. The horizontal axis represents stages of kernel development starting with unfertilized ovules (point “0”), and 8, 12, 21, 25, and 35 days after pollination (DAP). Endosperm and embryos were dissected from kernels. Note that embryo tissues were not dissected from 8 DAP kernels. Squares indicate endosperm; triangles indicate embryos.

FIG. 2 shows the pattern of paternal and maternal ZmFie1 allele expression in developing kernels. The graphs represent a size-dependent separation of the RT-PCR DNA fragments by the WAVE HPLC System. The larger fragments have a longer retention time on the DNASEP cartridge, which results in an accurate quantitative separation of the complex fragment mixture. Total RNA was isolated from 15 DAP kernels of selfed Mo17 and B73 lines and their reciprocal crosses. RT-PCR was performed with primers positioned around 12 nt deletions at 3′ UTR in Mo17 background (2A, 2B). The anonymous EST was used as a control for the expression of both maternal and paternal allele in the same samples of RNA (2C).

FIG. 3 shows that ZmFie2 Mo17 and B73 alleles are polymorphic by the MITE insertion at 3′ UTR. The position of a common forward primer F (exon 11) and the genotype-specific reverse primers (3′ UTR) are shown by arrows. DNA sequence of the MITE insertion into 3′ UTR of the ZmFie2 B73 allele is shown in 3B. The target site duplication is boxed. The 14 nt terminal inverted repeats are marked by arrowheads.

FIG. 4 shows the genomic structure of the ZmFie loci. 12 kb genomic segments of the ZmFie1 (A) and ZmFie2 (B) regions are shown. The predicted start and stop codons of ZmFie coding regions are indicated by ATG and TGA. The positions of nucleotides are relative to the translation start codon ATG. Exons are shown as tall vertical boxes, untranslated regions as shorter boxes, and introns as connecting double lines. The putative transcription and translation start sites are shown as bent arrows. Regions with homology to retrotransposons are stippled. The direct repeats positioned upstream of ZmFie2 are marked by large arrows.

FIG. 5 shows the 5′ upstream and coding sequence for the ZmFie1 gene sequence.

FIG. 6 shows the 5′ upstream and coding sequence for the ZmFie2 gene sequence.

FIG. 7 shows the distribution of the CpG and CpNpG methylation sites along the ZmFie genomic sequences. The graphs present the number of CpG or CpNpG sites per 100 nt. The start and stop codons are indicated by ATG and TGA. The CpG islands are marked by filled rectangles.

FIG. 8 shows a phylogenetic tree of plant FIE proteins.

FIG. 9 shows the distribution of Hpall restriction sites across the ZmFIE1 and ZmFIE2 genomic sequences.

FIG. 10 is a table of primers designed around clusters of Hpall sites to monitor cytosine methylation.

FIG. 11 shows single nucleotide polymorphisms (SNPs) present in exon 1 of B73 and Mo17 inbred lines.

### DETAILED DESCRIPTION OF THE INVENTION

Imprinting has been observed in eukaryotic cells of plants and mammals (Yoder and Bestor (1996) Biol. Chem. 377(10): 605-610). In humans and other mammals, normal imprinting underlies several fundamental cellular and developmental processes; thus, abnormal imprinting patterns are implicated in a wide variety of catastrophic human diseases. “Imprinting” is defined as an epigenetic modification of a specific parental allele of a gene, or the chromosome on which it resides, in the gamete or zygote, leading to differential expression of the two alleles in somatic cells of the offspring. That is, genomic imprinting is an epigenetic chromosomal modification in the germ line that leads to preferential expression of one of the two parental alleles in a parent-of-origin-specific manner. “Normal pattern of imprinting” means preferential expression of a single parental allele of an imprinted gene and/or preferential methylation of a single parental allele of an imprinted gene. “Loss of imprinting” or “LOI” means loss of a normal pattern of imprinting, i.e., the loss of preferential expression of a single parental allele of an imprinted gene and/or the loss of methylation of a single parental allele of an imprinted gene. LOI is exhibited by a variety of abnormal expression patterns. Such patterns include but are not limited to: equal expression of both alleles; significant (>5%) expression of the normally silent allele when the normal case is complete silencing of one allele; epigenetic silencing of the normally expressed copy of an imprinted gene; the absence of methylation of both alleles and/or the methylation of both alleles where the normal case is methylation of a single allele.

Imprinting is a developmental phenomenon wherein a gene in a gamete or zygote is modified such that preferential expression of a single parental allele occurs in the offspring. It has been theorized that “CpG islands” present within the gene are subject to methylation, which causes repression of one allele (Stoger et al. (1993) Cell 73:61-71). CpG islands are defined as sequences of 200 or more base pairs with a GC content greater than 0.5 and an observed-to-expected CpG dinucleotide content greater than 0.6 (Gardiner-Garden and Frommer (1987) J. Mol. Biol. 196:261-282). Allele-specific methylation of CpG islands is a feature of the inactive X chromosome (Yen et al. (1984) Proc. Natl. Acad. Sci. USA 81:1759-1763) and imprinted genes including H19, Snrpn, and lgf2r (Brandeis et al. (1993) EMBOJ 12:3669-3677; Shemer et al. (1997) Proc. Natl. Acad. Sci. USA 94:10267-10272; Wutz et al. (1997) Nature 389:745-749). Analysis of orthologous genomic domains of approximately 1 Mb in mouse and human identified nine conserved imprinted genes; in eight of these, two or more conserved CpG islands were found upstream of or within the gene. In contrast, six non-imprinted genes within the same region were associated with at most one CpG island (Onyango et al. (2000) Genome Research 10:1697-1710).

The present invention has identified CpG islands in plants and attributes differential expression of imprinted plant genes to CpG islands. Accordingly, the methods of the invention encompass the identification of imprinted plant genes by determining the presence of CpG islands. Where sequence information is available for a plant, the sequence can be searched for GC rich regions and further testing can be done to establish the location of CpG islands.

Methods for the determination of the pattern of imprinting are known in the art. It is recognized that the methods may vary depending on the gene to be analyzed. Generally, in methods for assaying allele-specific gene expression, RNA is reverse transcribed with reverse transcriptase, and then PCR is performed with PCR primers that span a site within an exon where that site is polymorphic (i.e., normally variable in the population), and this analysis is performed on an individual that is heterozygous (i.e., informative) for the polymorphism. One then uses any of a number of detection schemes to determine whether one or both alleles is expressed. Methods for the assessment of gene expression, allele-specific gene expression, and DNA methylation are encompassed. Additionally, direct approaches to identifying novel imprinted genes include: positional cloning efforts aimed at identifying imprinted genes near other known imprinted genes (Barlow et al. (1991) Nature 349:84-87); techniques comparing gene expression (Kuroiwa et al. (1996) Nat. Genet. 12:186-190); and restriction landmark genome scanning (Nagai et al. (1995) Biochem. Biophys. Res. Commun. 213:258-265). See also, Rainier et al. (1993) Nature 362:747-749; which teaches the assessment of allele-specific expression of IGF2 and H19 by reverse-transcribing RNA and amplifying cDNA by PCR using new primers that permit a single round rather than nested PCR; Matsuoka et al. (1996) Proc. Natl. Acad. Sci USA 93:3026-3030, which teaches the identification of a transcribed polymorphism in p57^{KIP2}; Thompson et al. (1996) Cancer Research 56:5723-5727, which teaches determination of mRNA levels by RPA and RT-PCR analysis of allele-specific expression of p57^{KIP2}; and Lee et al. (1997) Nature Genetics 15:181-185, which teaches RT-PCR SSCP analysis of two polymorphic sites. Such disclosures are herein incorporated by reference.

Direct approaches developed to identify novel imprinted genes include: positional cloning, which identifies imprinted genes near other known imprinted genes (Barlow et al. (1991) Nature 349:84-87); comparing gene expression in parthenogenetic embryos to that of normal embryos (Kuroiwa et al. (1996) Nat. Genet 12:186-190); and restriction landmark genome scanning (Nagai et al. (1995) Biochem. Bionhys. Res. Commun. 213:258-265). The last approach comprises analysis of clonality in tumors by assessing DNA methylation near a heterozygous polymorphic site (Vogelstein et al. (1985) Science 227:642-645).

As noted above, a distribution of CpG islands within genes can be used as a predictive tool for genes regulated by imprinting. To date, imprinted genes in plants are important components of regulation of endosperm size and growth. Thus, the methods of the invention can be used to identify genes involved in endosperm development. In particular, the invention can be used as a predictive tool for plant genes, dicot and monocot genes, particularly maize genes, that are regulated by imprinting.

It is also recognized that the CpG islands of the invention may be used to silence paternally transmitted genes. In this manner, DNA constructs comprising at least two CpG islands will be operably linked with a coding sequence and a promoter that is expressed in plants.

A number of promoters can be used in the practice of the invention. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Chemically-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112 (2):525-535; Canevascini et al. (1996) Plant Physiol. 112 (2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference.

Examples include, for dicotyledonous plants, a bean β-phaseolin promoter, a napin promoter, a β-conglycinin promoter, a cruciferin promoter, and a soybean lectin promoter. For monocotyledonous plants, promoters useful in the practice of the invention include, but are not limited to, cZ19B1 (maize 19 kDa zein), milps (myo-inositol-1-phosphate synthase), celA (cellulose synthase) (see WO 00/11177, herein incorporated by reference), a maize 15 kD zein promoter, a 22 kD zein promoter, a 27Kd γ-zein promoter (such as gzw64A promoter, see Genbank Accession #S78780), a waxy promoter, a shrunken-1 promoter, a globulin 1 promoter (See Genbank Accession #L22344), an Itp2 promoter (Kalla, et al., Plant Journal 6:849-860 (1994); U.S. Pat. No. 5,525,716), cim1 promoter (U.S. Pat. No. 6,225,529), maize end1 and end2 promoters (See U.S. patent application Ser. No. 09/383,543, filed Aug. 26, 1999, and Ser. No. 10/310,191, filed Dec. 4, 2002), and the shrunken-2 promoter. See also U.S. Pat. Nos. 6,407,315 and 6,403,862. However, other promoters useful in the practice of the invention are known to those of skill in the art such as nucellain promoter (See C. Linnestad, et al., Plant Physiol. 118:1169-80 (1998)), kn1 promoter (See S. Hake and N. Ori, B8: INTERACTIONS AND INTERSECTIONS IN PLANT PATHWAYS, COEUR D'ALENE, IDAHO, KEYSTONE SYMPOSIA, Feb. 8-14, 1999, at 27.), and F3.7 promoter (Baszczynski et al., Maydica 42:189-201 (1997)). Spatially acting promoters such as glb1, an embryo-preferred promoter; or gamma zein, an endosperm-preferred promoter, or BETL1 (See G. Hueros, et al., Plant Physiology 121:1143-1152 (1999)), are particularly useful. The use of temporally acting promoters is also contemplated by this invention. Promoters that act from 0-25 days after pollination (DAP) are preferred, as are those acting from 4-21, 4-12, or 8-12 DAP. In this regard, promoters such as cim1 and Itp2 are preferred. Particularly preferred promoters include maize zag2.1 (GenBank Accession X80206), maize zap (see U.S. Provisional Patent Application No. 60/364,065), maize ckx1-2 promoter (see U.S. Patent Publication 2002-0152500 A1), maize end2 (see U.S. Pat. No. 6,528,704, and also U.S. patent application Ser. No. 10/310,191, filed Dec. 4, 2002), and maize lec1 (see U.S. patent application Ser. No. 09/718,754, filed Dec. 27, 2002).

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture; Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and resulting plants having desired expression of the subject phenotypic characteristic may be identified. Two or more generations may be grown to ensure that the desired expression of the subject phenotypic characteristic is stably maintained and inherited and then seeds harvested to ensure that desired expression of the subject phenotypic characteristic has been achieved.

The following examples are offered by way of illustration, not by way of limitation.

### EXPERIMENTAL

Introduction

A fundamental problem in biology is to understand how fertilization initiates reproductive development. In flowering plants, the female gametophyte, or embryo sac, is composed of egg, central, synergid, and antipodal cells. Double fertilization triggers development of the egg into a diploid embryo and development of the central cell into a triploid endosperm. In sexually-reproducing plants, the embryo sac never develops into seed without fertilization. In asexually-reproducing apomictic plants, the egg cell develops parthenogenetically without fertilization to produce the embryo, but in many species the endosperm development may still require fertilization (non-autonomous apomicts) (Grimanelli et al. (2001) Trends Genet. 17(10):597-604).

A number of mutants that initiate fertilization independent seed (FIS) development have been isolated in Arabidopsis (Ohad et al. (1996) Proc. Natl. Acad. Sci. USA 93(11):5319-5324; Chaudhury et al. (1997) Annu. Rev. Cell Dev. Biol. 17:677-699). These mutants uncouple seed development from the fertilization process and display some characteristics of apomixis, such as autonomous endosperm development. A mutational approach has revealed three genes with similar FIS phenotypes: FIS1/MEDEA, which is related to the Polycomb group (PcG) protein EZ (enhancer of Zest) of Drosophila (Grossniklaus et al. (1998) Science 280:466-450; Luo et al. (1999) Proc. Natl. Acad. Sci. USA 94(8):4223-4228); FIS2, which is a C₂H₂ Zinc Finger transcriptional regulator that may have a similar function to Hunch back protein of the Drosophila PcG complex (Luo et al. (1999) Proc. Natl. Acad. Sci. USA 94(8):4223-4228); and FIS3/FIE, which is a homologue of the PcG protein ESC (extra sex combs) (Ohad et al. (1999) Plant Cell 11:407-416). Polycomb group proteins are conserved among eukaryotes and are involved in the repression of homeotic genes during early development in flies and mammals. One could speculate that FIS genes define a PcG-like complex in plants that suppresses the development of the endosperm in the absence of fertilization (Grossniklaus et al. (1998) Science 280(5362):466-450; Luo et al. (1999) Proc. Natl. Acad. Sci. USA 94(8):4223-4228; Ohad et al. (1999) Plant Cell 11:407-416).

The Arabidopsis model provides candidate genes for revealing similar pathways in other plants. A search of the homologues in a proprietary maize EST (Expressed Sequencing Tags) database identified two maize genes, ZmFie1 and ZmFie2 (see WO 01/16325, herein incorporated by reference). The putative FIE maize proteins share 57-68% identity with the Arabidopsis FIE protein. FIS2/3 genes do not demonstrate such a remarkable conservation. A duplication of the maize Fie gene raises a question about their functional redundancy. The two ZmFie genes show a different pattern of expression in vegetative and reproductive tissues, but they may have overlapping function in the developing kernels.

In this example, the expression of two maize Fie genes in developing kernels has been analyzed by several different methods, which lead to the conclusion that the two ZmFie genes may have nonredundant functions. Based on the expression pattern and a temporal type of imprinting, ZmFie2 is likely to be a functional homologue of the Arabidopsis FIE gene and most likely is involved in the repression of endosperm development before pollination. The expression of ZmFie1 is triggered in endosperm after pollination, which implies no repressive function in the embryo sac before pollination, but reveals a new endosperm-specific FIE function in maize. Only the maternal ZmFIE1 allele is expressed during kernel development, implying a strong regulation by imprinting. Based on the genomic sequences of ZmFIE genes, different models for temporal and permanent types of imprinting are proposed. Thus far the ZmFIE1 gene is found only in maize, which is likely to be a consequence of its allotetraploid origin.

Experimental Procedures

RNA Gel Blot Analysis.

To analyze ZmFIE expression in developing kernels, mRNA was isolated from non-pollinated ovules at silking and from kernels at 3, 6, 9, 12, and 15 days after pollination (DAP). Total RNA was extracted from 1 g of material using a hot phenol extraction procedure and a selective precipitation with 4 M LiCl to remove traces of DNA and small RNA species (Verwoerd et al. (1989) Nucleic Acids Res. 17:2362; Brugiere et al. (1999) Plant Cell 11:1995-2012). For each time point, kernels were collected from two ears harvested from two different plants (replications) from either the B73 or Mo17 inbred lines. RNA was quantified using a spectrophotometer at 260 nm. Poly(A) was prepared from total RNA (400 μg) using the Oligotex™ poly(A) purification kit (Qiagen). For gel blot experiments, poly(A) RNA enriched samples were prepared as described by Becker et al. (1993) Methods Enzymol. 218:568-587. Three μg of polyA RNA were loaded in each lane. Electrophoretic separation was performed on 1.5% agarose gels containing 5% (v/v) of a solution of 37% formaldehyde in Mops buffer (0.02 M Mops, pH 7.0, 5 mM sodium acetate, and 1 mM EDTA). Gels were blotted onto a nylon membrane (Roche Molecular Biochemicals) using TurboBlotter (Schleicher & Schuell), with 20×SSC (1×SSC is 150 mM NaCl, 15 mM sodium citrate) as transfer buffer. Blots were probed with ³²P-labeled 300 bp fragments of ZmFIE1 or ZmFIE2 cut from the 3′ UTR of the appropriate ETS clones. The fragment sequences shared no homology, which avoided cross-hybridizations. Actin probe was used as a loading control.

Distinguishing ZmFie mRNAs in Reciprocal Crosses.

Reciprocal crosses between B73 and Mo17 inbred lines were performed, and F1 kernels were sampled at 2, 5, 10, and 15 days after pollination (DAP). Total RNA was isolated and reverse PCR reactions were performed with “Superscript kit.” The PCR product differed between B73 and Mo17 alleles in a 12 nt deletion. PCR product was separated on HPLC WAVE machine to distinguish between B73 and Mo17 alleles.

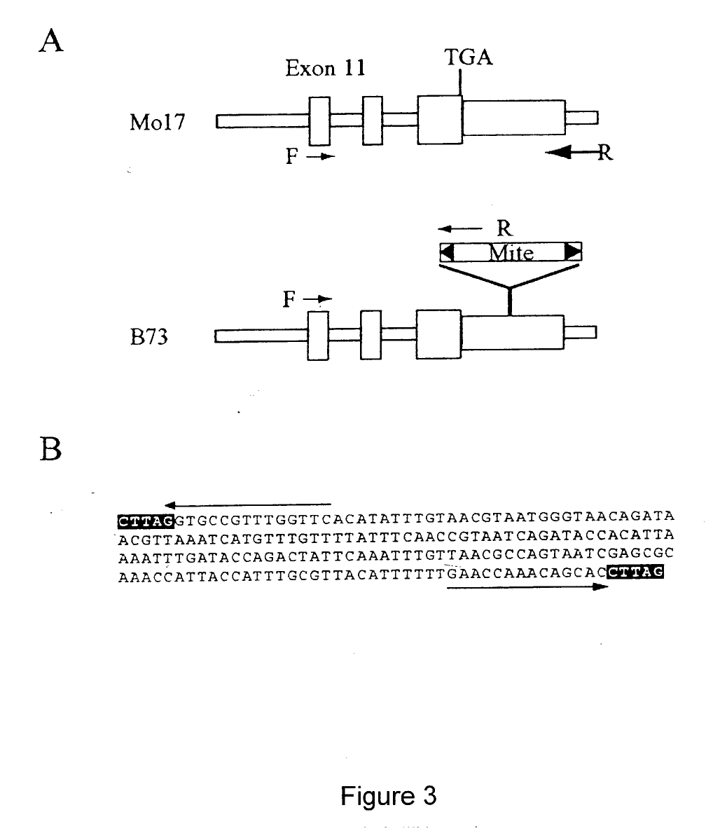

Primers to amplify ZmFIE2 were designed based on the MITE insertion in the B73 ZmFIE2 allele. In B73 background, ZmFIE2 polyA transcripts are terminated in the middle of this insertion. In Mo17 background, ZmFIE2 polyA transcripts are terminated within genomic sequence with no homology to MITE insertion. (See FIG. 3A.) The forward primer positioned in exon eleven, 5′-CGTGAAGGCAAAATCTACGTGTGG-3′, (SEQ ID NO: 2) is common for both genotypes. The reverse primer 5′-CATTACGTTACAAATATGTGAACCAAACG-3′ (SEQ ID NO: 3) is specific for the B73 allele; reverse primer 5′-CAGAACAAACAGATGACAACGGTTCCCAAAG-3′ (SEQ ID NO: 4) is specific for the Mo17 allele. This primer combination allows for monitoring of B73 and Mo17 ZmFIE2 allele expression in developing kernels of the reciprocal crosses by RT-PCR.

In Situ Hybridization.

To determine expression patterns of ZmFIE genes in maize, in situ hybridization was performed using the protocol of Jackson (1991) in In situ Hybridization in Plants, Molecular Plant Pathology: A Practical Approach, ed. Bowles et al. (Oxford University Press, England), pp. 63-74. Sense and antisense mRNA probes of 300 bp corresponding to the 3′ UTR of ZmFIE genes were labeled non-isotopically with digoxigenin-UTP by in vitro transcription with T7 and T3 RNA polymerases (Roche Molecular Biochemicals). Probes were hybridized with fixed sections of maize tissues from ovules at silking, and kernels at 5, 8, and 12 DAP. Following extensive washing to remove unbound probe, signal was detected with anti-DIG-antibodies conjugated with alkaline phosphatase to mediate color reaction (Roche Molecular Biochemicals) that leads to a purple-blue precipitate in the cells that contain mRNA. ZmFIE mRNAs were detected specifically with the antisense probe; the sense probe did not hybridize, therefore serving as a negative control.

Cloning and Sequencing of ZmFIE Genomic Fragments.

BAC genomic libraries were screened with ZmFIE1 and ZmFIE2 ESTs. Five BAC clones per each gene were identified and confirmed by Southern hybridization. HindIII and EcoR1 BAC fragments subcloned into vector BluescriptII (KS) (Stratagene) were hybridized with ZmFIE probes, and positive clones were sequenced.

DNA Sequence Analysis.

DNA assembly was performed using the Sequencher program (Genecode, Ann Arbor, Mich.). BLAST search of GenBank was used for sequence annotation. Sequence analysis was performed with GCG® programs (Accelrys, Inc., San Diego, Calif.).

Nucleotide Sequence Accession Numbers.

The sequences have been deposited in the GenBank database under Accession No. AY061964 (ZmFie1 genomic locus), and AY061965 (ZmFie2 genomic locus).

### Example 1

### Maize FIE (Fertilization Independent Endosperm) Homologues: Two Related Genes with Distinct Expression Patterns

Results

Expression of ZmFIE Genes in Developing Kernels.

ZmFie genes have a different pattern of expression in vegetative and reproductive tissues. Expression of ZmFIE1 was detected only in developing kernels, not in vegetative tissues. Conversely, ZmFIE2 expression was found in all tissues tested. If these genes participate in repression of embryo sac development before fertilization in a manner similar to the Arabidopsis FIE homologue, they should be expressed in the ovules before fertilization. To understand the function of both genes, their expression in ovules and developing kernels was detected by mRNA gel blot experiments, gene expression analysis by massively parallel signature sequencing (MPSS) (Brenner et al. (2000) Nat. Biotechnol. 18:630-634), and by in situ hybridization.

For RNA gel blot experiments, mRNA was isolated from non-pollinated ovules and from developing kernels at 3, 6, 9, 12, and 15 days after pollination (DAP). ZmFIE1 mRNA is not detected in ovules and 3 DAP kernels. It appears first in 6 DAP kernels, reaching a maximum of expression in 9 DAP kernels, and gradually declines at later stages. The expression pattern of ZmFIE2 is very different: mRNA is detected in ovules and all stages of developing kernels, but declines after 6 DAP. RNA gel blot experiments demonstrate a low-abundance of ZmFIE2 mRNA, compared to ZmFIE1 mRNA, which shows significantly higher expression.

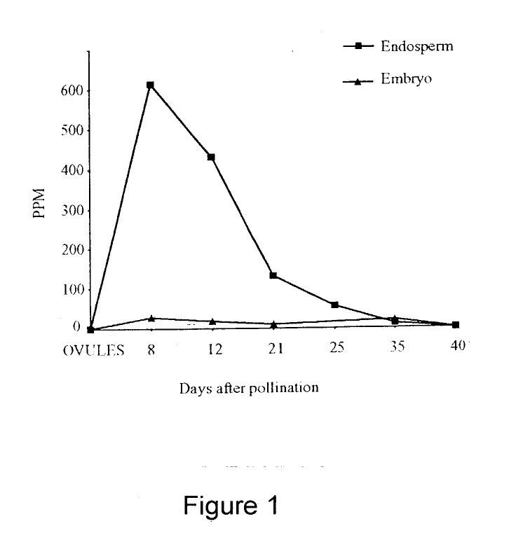

To achieve a more sensitive assay of ZmFIE expression, these cDNA sequences were searched with a BLAST algorithm against the gene expression database generated by the MPSS method from different maize tissues. Massively parallel signature sequencing (MPSS) generates 17-mer sequencing tags of millions of cDNA molecules, which are in vitro cloned on microbeads (Brenner et al. (2000) Nat Biotechnol. 18:630-634). The technique provides an unprecedented depth and sensitivity even for messages that are expressed at very low levels. MPSS is based on the Dpnll (GATC) restriction site availability in cDNA templates. If the site is absent, the 17-mer tags are not generated. ZmFie2 does not have the appropriate Dpnll site and is not suitable for MPSS analysis. For this reason, only ZmFIE1 tags were found. Distributions of the ZmFIE1 tags in MPSS experiments are shown in FIG. 1. No tags were detected in mRNA isolated from ovules. Thus, if ZmFIE1 were transcribed in ovules, it would produce less than one mRNA molecule per 10⁶ total mRNA molecules. At 8 DAP, the number of tags is about 600 PPM (particles per million), gradually decreasing at later stages and reaching 20 PPM at 35 DAP. No tags are found in 40 DAP kernels. This trend is in complete agreement with mRNA gel blot experiments and RT-PCR (data not shown). The second important observation from MPPS experiments is the expression of ZmFie1 in the developing endosperm. Embryo and endosperm were dissected for MPSS experiments from kernels as early as 10 DAP. At this stage, ZmFIE1 expression is approximately 20-30 times higher in endosperm than in embryo. MPSS analysis strongly suggests that transcription of ZmFIE1 is activated in developing kernels approximately 5-6 days after pollination, predominantly in endosperm.

Because this type of analysis is not available for ZmFIE2, in situ hybridization was performed. Longitudinal sections of B73 ovules and kernels at 2, 5, 8 and 15 DAP were prepared and hybridized with antisense RNA probes, and with sense RNA probes as a negative control. The sense probe revealed no background signals, and images are not shown. ZmFIE2 antisense probes gave a signal in the embryo sac of the mature ovules at silking. At 2 DAP, zygotes had a significantly increased signal compared to ovules, indicating that ZmFIE2 transcription is activated de novo, and the signal intensity may not be explained by the pre-existing maternal RNA. In kernels at 5 DAP, the most intense signal appeared in the embryo-surrounding region and on the periphery of the developing endosperm. At the later stage of 15 DAP, the signal persists in the embryo and is not detectable in the endosperm. It shows also the clear pattern of an axis polarity, being more intensive in the areas of leaves and root primordia.

In summary, ZmFIE2 gene is expressed in the embryo sac before pollination and in developing embryo after fertilization, as well as in vegetative tissues. This pattern of expression is very similar to that observed for Arabidopsis FIE, but very different from that observed for ZmFIE1.

Pattern of Maternal and Paternal ZmFie Allele Expression During Kernel Development.

The Arabidopsis FIE gene demonstrates a parent-of-origin effect on seed development, suggesting that only the maternal FIE allele is essential, whereas the paternal FIE allele plays no role in seed development (Yadegari et:al. (2000) Plant Cell 12:2367-2382; Luo et al. (2000) Proc. Natl. Acad. Sci. USA 97(19): 10637-10642). Current evidence supports the model that the FIE gene is an imprinted gene, in which the maternal allele is expressed and the paternal allele is silenced during seed development (Yadegari et al. (2000); Luo et al. (2000) Proc. Natl. Acad. Sci. USA 97(19):10637-10642). To understand whether the maize FIE homologues are regulated by imprinting in the same manner as the Arabidopsis FIE gene, the paternal- and maternal-specific FIE mRNA levels were measured in developing kernels.

To distinguish maternal and paternal ZmFIE mRNAs, the insertion/deletion sequencing polymorphism was identified in both ZmFIE1 and ZmFIE2 genes in inbred lines Mo17 and B73. Reciprocal crosses were performed between B73 and Mo17 lines, and kernels were collected at 2, 5, 10, 15, and 16 DAP. Ovules and selfed kernels from both inbred lines were sampled at 11 DAP as controls. Total RNA was extracted from the whole kernels.

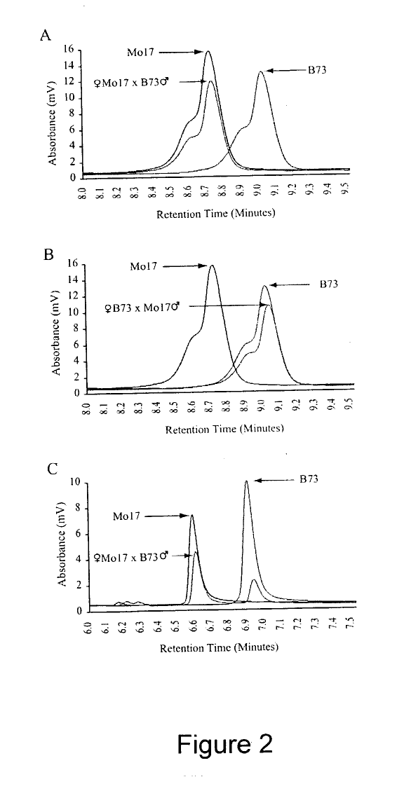

Mo17 and B73 ZmFIE1 alleles are different by a 12 nt insertion/deletion in the 3′ UTR. The reverse and forward primers were designed around this indel to produce the 300 bp RT-PCR product, which was separated on D-HPLC column by WAVE machine. As shown in FIGS. 2A and 2B, only maternal ZmFIE1 RNAs were detected in reciprocal crosses in 15 DAP kernels. No detectable level of the paternal RNA was found at early stages (data not shown). The same set of RNAs was used with an anonymous gene as a control for bi-allelic expression (FIG. 2C). The paternal allele of a control non-imprinted gene was detected in 5 DAP kernels and all later stages, confirming that the paternal gene is expressed in kernels. Thus, the ZmFIE1 paternal allele undergoes transcriptional silencing in developing kernels, and this gene is regulated by imprinting. As noted above, ZmFIE1 is expressed predominately in endosperm; this is in agreement with previous reports that all known imprinted genes in plants are expressed in triploid endosperm. Thus far, imprinting has not demonstrated for genes expressed in diploid tissues.

A different strategy was used for monitoring allelic expression of ZmFie2. ZmFie2 genomic sequence from inbred B73 contains the 185 nt MITE insertion at 3′ UTR, which is not present in the Mo17 allele (FIG. 3A). The insertion is flanked by 15-nt inverted repeats and creates the 5 nt direct target duplication (FIG. 3B). These features are typical for MITE elements, which are very abundant components of the maize genome (Wessler (2001) Plant Physiol. 125(1):149-51). In B73, ZmFIE2 polyA transcripts are terminated in the middle of the MITE insertion. In Mo17 background, ZmFIE2 polyA transcripts are terminated within genomic sequence with no homology to MITE. The MITE sequence was used to design allele-specific primers to discriminate between B73 and Mo17 ZmFIE2 mRNAs (FIG. 3A).

The forward primer, F, designed for exon 11, is common for both genotypes. The reverse primers, R, are genotype specific. The primer combinations are highly allele-specific; no RT-PCR products are found in RNA samples from ovules or selfed homozygous kernels. The primers allow monitoring of the expression of maternal and paternal ZmFIE2 alleles in developing kernels. Maternal allele expression was detected at all stages in both reciprocal crosses, being more abundant in 2 DAP zygotes. These results are in agreement with the in situ hybridization data, which demonstrated an increased ZmFIE2 expression in 2 DAP zygotes in the embryo-surrounding region. Paternal allele expression is delayed up to 10 DAP, but at later stages, both maternal and paternal alleles are expressed. Delayed expression, but not a complete silencing, of the paternal allele is a feature of the Arabidopsis FIE gene. As mentioned previously, the ZmFIE1 gene undergoes permanent silencing of the paternal allele, demonstrating a different type of imprinting.

Genomic Structure of ZmFIE Loci.

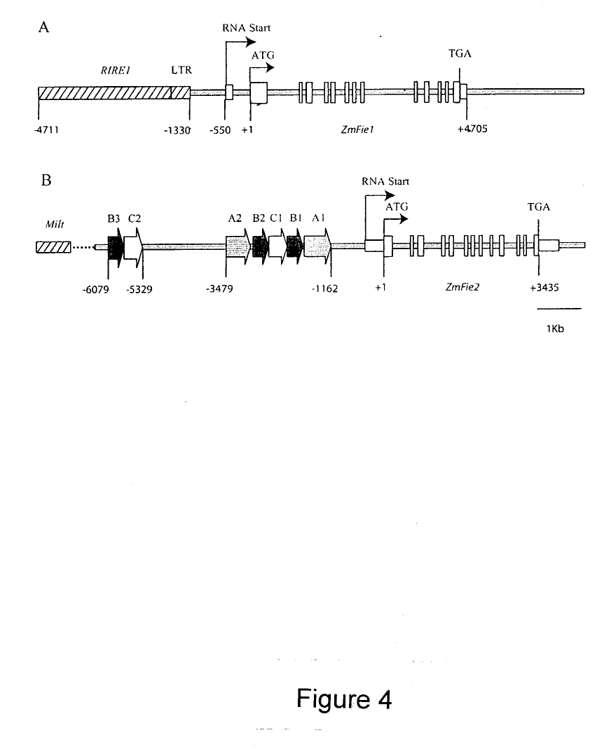

The transcriptional pattern of ZmFIE genes is very different with respect to tissue specificity, efficiency, and imprinting. ZmFIE1 is expressed only in developing kernels at a relatively high level, and with a permanent silencing of the paternal allele. Conversely, ZmFIE2 is expressed in vegetative and reproductive tissues, showing a very low level of expression in developing kernels, with delayed paternal allele expression. To reveal the molecular mechanisms underlying the different patterns of ZmFIE expression, the genomic loci of both genes have been sequenced. Genomic BAC libraries were screened with ZmFIE1 and ZmFIE2 cDNAs. Five BACs were identified for each gene covering the overlapping regions (about 250 kb). Approximately 12-kb segments carrying ZmFIE genes have been subcloned and sequenced (FIGS. 4A and 4B). The positions of nucleotides are relative to the translation start site, ATG (+1). (The transcription start site is used more often as a reference point, but it is not identified precisely for FIE transcripts.)

The coding regions of both genes downstream of the translation start site, ATG, possess 13 exons, which are identical in size between the two genes, except for the first and last exons where initiation and termination of transcription occur. The number and sizes of the protein coding exons are also identical to the Arabidopsis FIE gene (GenBank Accession No. AF129516). The intron sequences vary in length and do not share a significant homology between ZmFIE1 and ZmFIE2 and Arabidopsis. However, ZmFIE1 demonstrates a unique feature among the FIE family, the presence of a 290 bp intron, located in the 5′ UTR, just 6 nucleotides upstream from the ATG codon (−6 and 390). The first exon and intron are very often required for high level expression of the reporter, which may be a result of the increased level or stability of the mature cytoplasmic mRNA constructs (Kim and Guiltinan (1999) Plant Physiol. 121(1):225-236); Clancy et al. (1994). It is very likely that the 5′ UTR intron of ZmFIE1 plays a regulatory role or determines the tissue specificity of FIE1 protein expression.

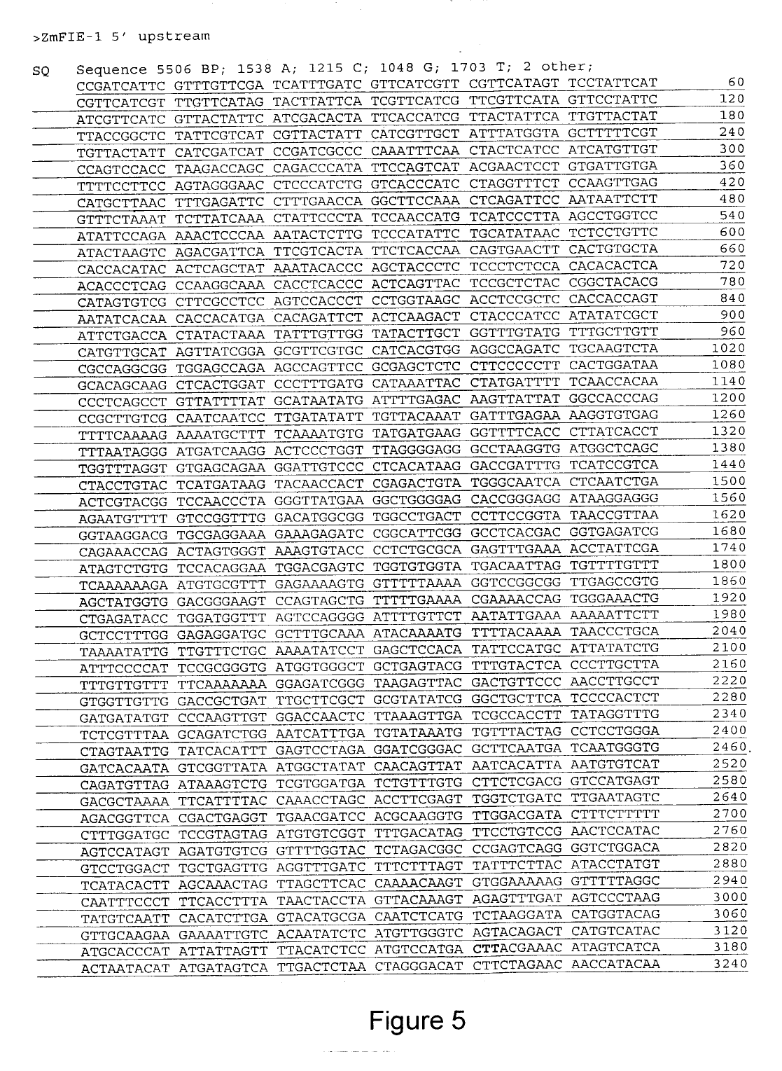

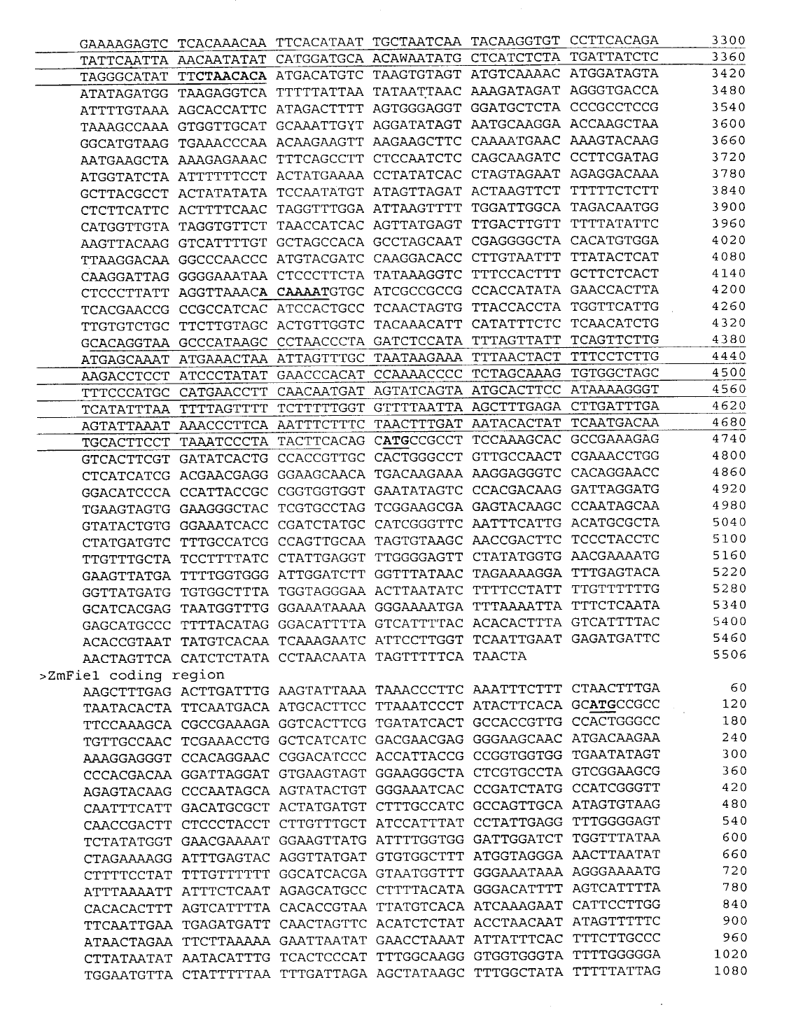

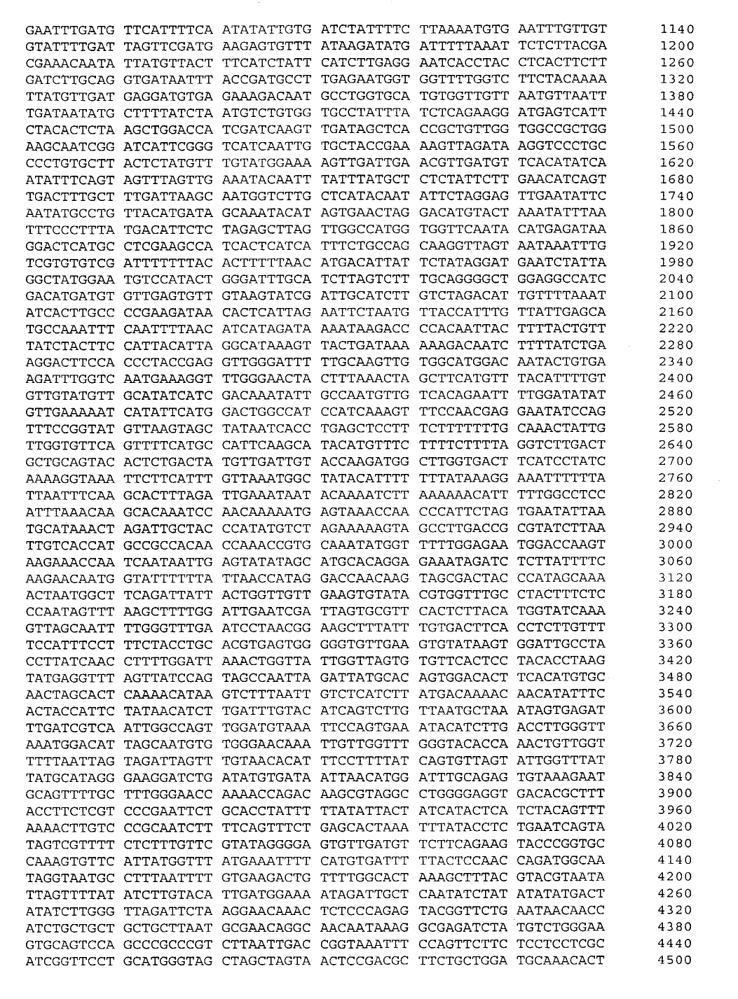

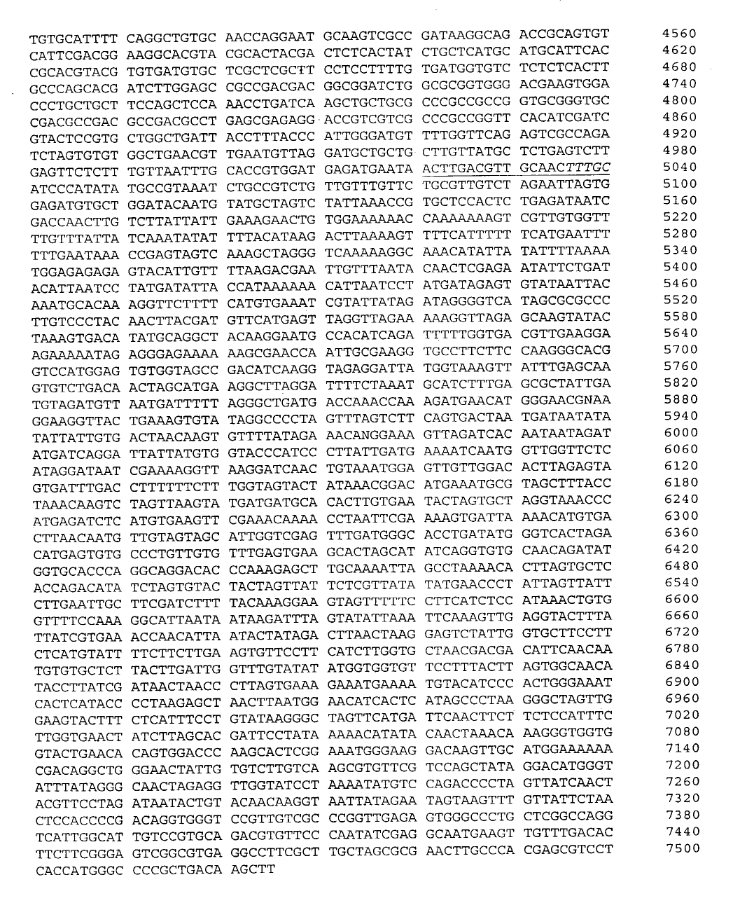

The 5′ upstream regions of the two genes are very different. The size of the putative promoter region of the ZmFIE1 gene is estimated to be about 900 nt, between the RNA start of the longest EST (Accession No. AY061964) and the retrotransposon RIRE LTR (FIG. 4A; FIG. 5). Dot plot analysis (data not shown) does not reveal any repeats as far as 5 kb upstream of the RIRE retrotransposon. Repeats are commonly speculated to be involved in imprinting (Alleman and Doctor (2000) Plant Mol. Biol. 43:147-161). However, this analysis indicates that this is very unlikely to be the case for the imprinting mechanism of the ZmFIE1 gene.

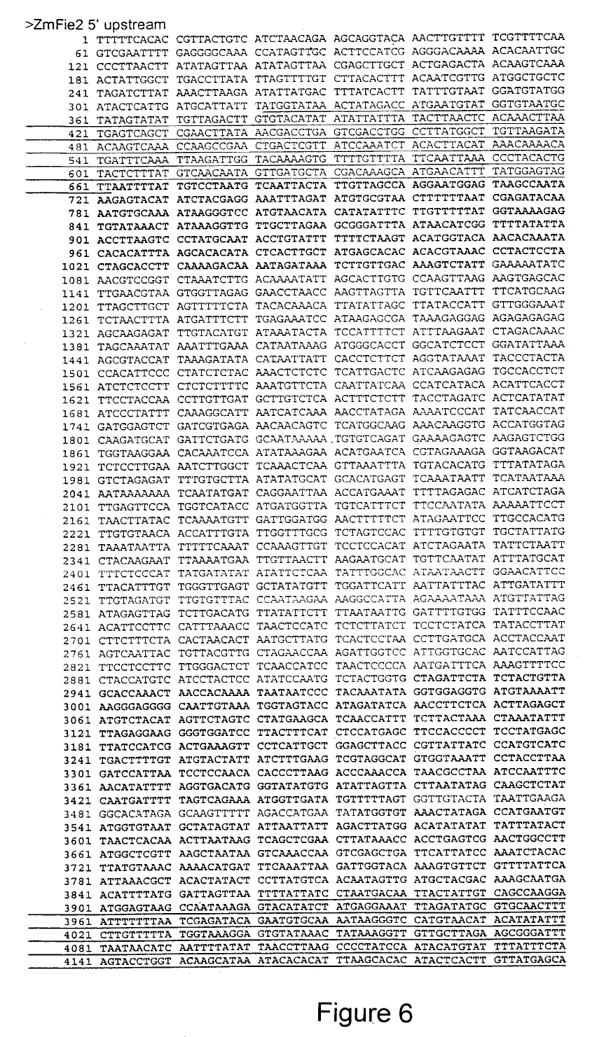

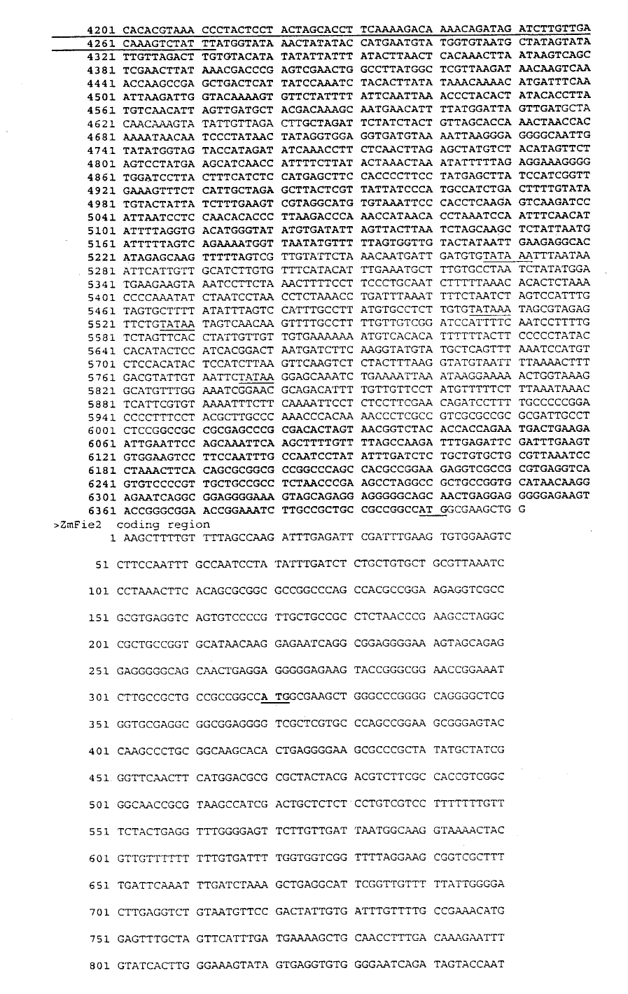

The 5′ upstream region of the FIE2 gene is about 6 kb long as estimated between the transcription start site of the ZmFIE2 longest cDNA (Accession No. AY061965) and the retrotransposon MILT LTR. The extensive BLAST search of this sequence against the public and proprietary databases did not show any homology to known sequences, suggesting that the 6 kb 5′ upstream region of the ZmFIE2 gene is its unique integral part. Dot plot analysis (not shown) revealed the complex pattern of repeats positioned along the 6 kb upstream region (FIG. 4B; FIG. 6). The sequence between −1161 and −3479 consists of three types of repeats, named A, B, and C. Repeats form a 2.6 kb symmetrical structure having the following order: A1-B1-C1-B2-A2. The B3 and C2 types are repeated again (−5328 to −6077) forming one more cluster. Repeats A1-A2 are 550 nt long with 95% homology; B1-B2-B3 are 350 nt long with 94% homology, and C1-C2 are 420 nt long with 93% homology (FIG. 6). Repeats do not share any homology or features of the transposable elements. They form a unique configuration and may be considered as a potential cis-regulating element of the ZmFIE2 gene. The basal promoter of the ZmFIE2 gene is estimated to be about 768 bp if framed between −393 and −1161, which marks the transcription start of the longest EST and the beginning of the B2 repeat.

The CG Composition of the ZmFIE Genes in Relation to Imprinting.

As discussed above, ZmFIE expression is regulated by imprinting but in a different temporal fashion. The paternally derived ZmFIE1 allele is permanently silenced during kernel development. Expression of ZmFIE2 undergoes less stringent temporal imprinting, because the paternal allele is reactivated later in kernel development (after 10 DAP). It has been widely speculated that imprinting is mediated by DNA methylation. CpG island methylation may be a key molecular mechanism of imprinting (Wutz et al. (1997) Nature 389(6652):745-749; Thorvaldsen et al. (1998) Genes Dev. 12(23):3693-3643; Reik and Dean (2001) Electrophoresis 22(14):2838-2843). Recently a two-island rule was proposed to define genes regulated by imprinting (Onyango et al. (2000) Genome Res. 10(11): 1697-1710). In this reference, comparative analysis of human and mouse imprinted genes revealed that two or more CpG islands are associated with imprinted genes, while at most one GpG island is associated with nonimprinted genes. The CpG islands were defined in this reference as sequences of about 200 bp with a GC content >50% and an observed-to-expected CpG content >60%. These criteria were applied for searching for CpG islands along the FIE loci.

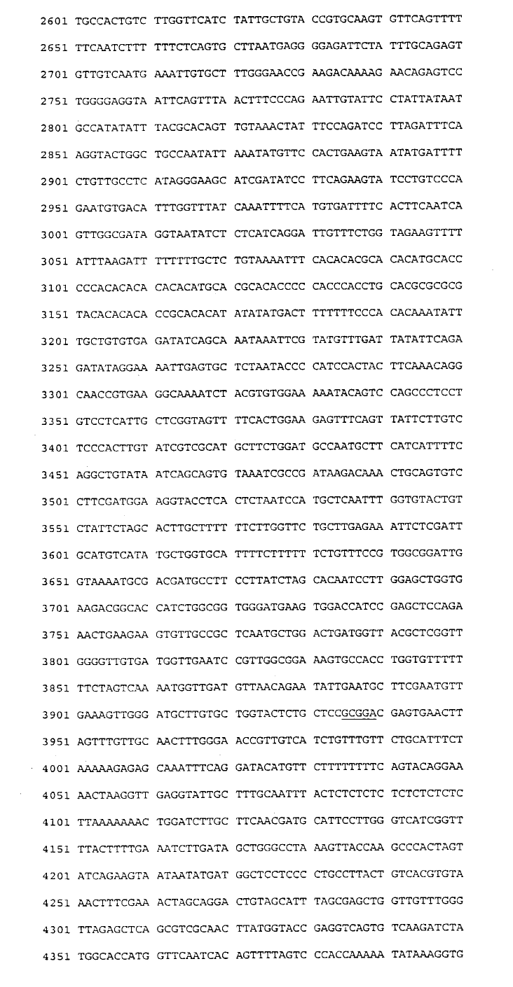

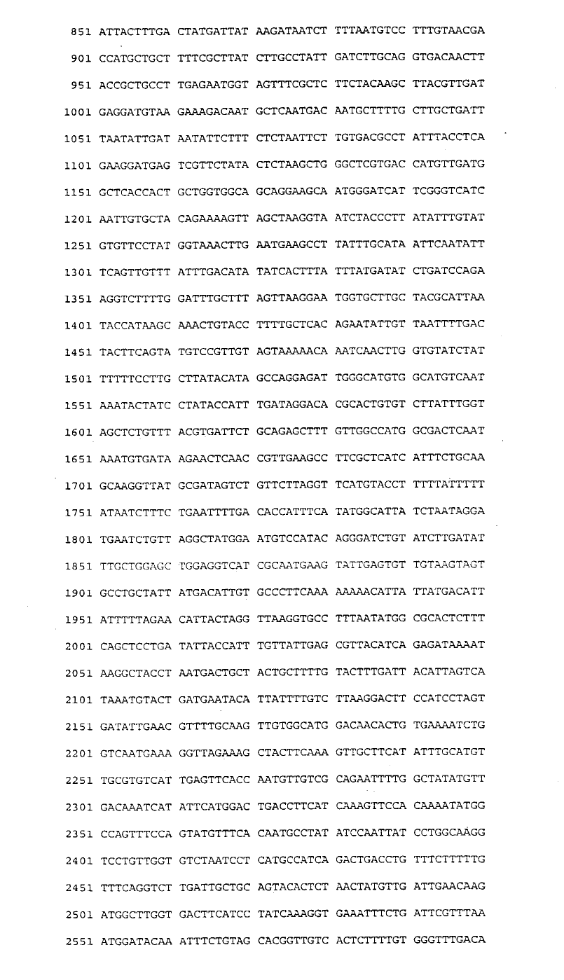

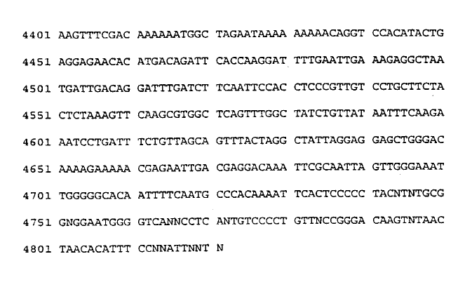

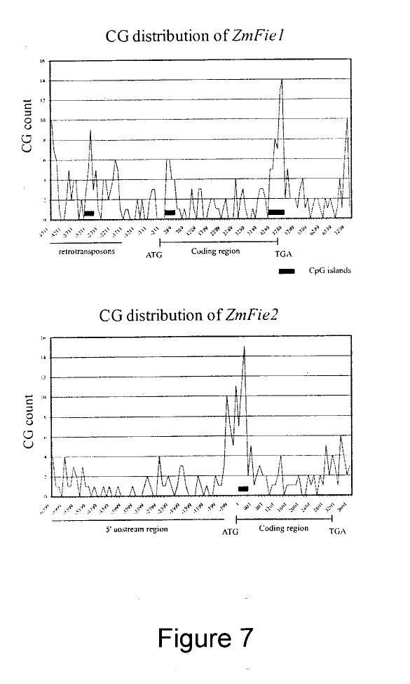

This analysis revealed three CpG islands within the ZmFIE1 locus. One island is located between −2968 and −3219 (FIG. 7), which corresponds to the retrotransposon segment and very likely is irrelevant to regulation of ZmFIE1. The other two islands are located within the ZmFIE1 coding region, which agrees with the two-island rule. The first of these two CpG islands is 252 bp and is positioned between +87 and +374, just downstream of the ATG codon. The second of these two CpG islands is 572 bp long and is located at the 3′ end of the gene, between +4315 and +4886, covering the last two introns and exons.

Only one CpG island is present in the ZmFie2 locus, at position −231 to +88, around the ATG codon (FIG. 7). This agrees with the definition of non-imprinted genes, which are associated with at most one CpG island (Onyango et al. (2000) Genome Res. 10(11):1697-1710).

These data suggest that the imprinting mechanism of ZmFIE1 is very likely associated with DNA methylation of two CpG islands. The delayed expression of the paternal ZmFIE2 allele, which could be considered as a temporal imprinting, is not associated with DNA methylation. The complex repetitive structure of the 5′ upstream region may be responsible for this type of imprinting.

Phylogenetic Analysis of Plant FIE Proteins.

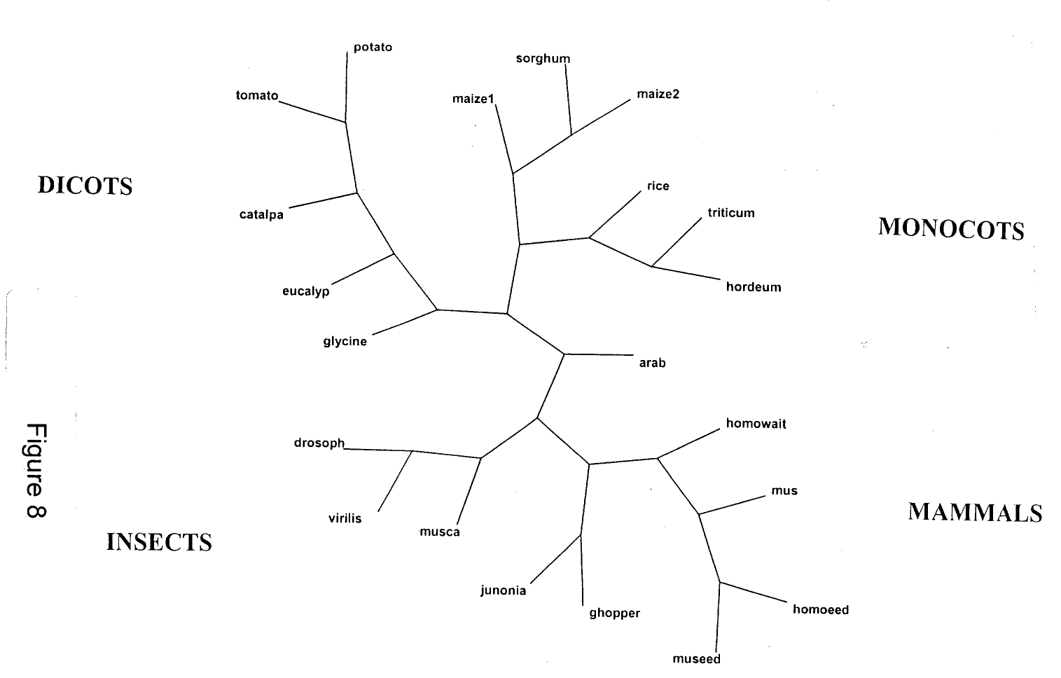

ZmFIE1 and ZmFIE2 genes are mapped to chromosome 4 (bin 4.05) and chromosome 10 (bin 10.3). These regions are duplicated in the maize genome (Helentjaris (1995) Maize Newsletter 69:67-81; Gaut and Doebley (1997) Proc. Natl. Acad. Sci USA 94(13):6809-6814). It is very likely that the two ZmFIE genes are due to the allotetraploid origin of the maize genome (Gaut and Doebley (1997), supra. Presence of two FIE genes in the maize genome raises the question whether two FIE genes exist in other species as well. A search by TBLASTX of the public EST database reveals accession numbers for 11 species, and putative FIE proteins were reconstructed. The FIE protein belongs to the Polycomb Group (PcG) proteins, which include the Drosophila extra sex combs (ESC), and mammalian embryonic ectoderm development proteins (EED). To make the phylogenetic analysis more robust, the PcG proteins from five insect and two mammalian species were included. A phylogenetic tree was constructed using the PAUP program (FIG. 8). The phylogenetic tree forms four major clades corresponding to mammals, insects, monocots, and dicots. The Arabidopsis FIE protein is positioned apart, reflecting the absence of the protein from related species.

So far all analyzed plant species show the presence of the one putative FIE protein. The phylogenetic tree demonstrates that the sorghum FIE and ZmFIE2 proteins are more closely related to each other than ZmFie1 protein. Thus, a ZmFie1 analog has not yet been found. This does not prove the absence of homologs to ZmFIE1 in other species, but the probability is very high that FIE1 is unique to the maize genome.

Discussion

ZmFIE Genes Are Differentially Expressed.

In understanding the role of ZmFIE genes, it is crucial to know in which tissues and cells these loci are active and whether two genes are active in the tissues at the same developmental times. The Arabidopsis single FIE gene (AtFIE) is expressed in many tissues, both reproductive and vegetative, indicating that this FIE protein may have multiple functions during plant development. AtFIE is expressed in the embryo sac before fertilization, and its expression continues in the embryo and endosperm after fertilization (Ohad et al. (1999); Luo et al. (2000) Proc. Natl. Acad. Sci. USA 97(19):10637-10642) Loss-of-function alleles of AtFIE demonstrate pleiotropic phenotypes, including initiation of endosperm development without fertilization, embryo abortion at early stages, premature flowering by seedling shoots, and flower-like structures along the roots and hypocotyls (Ohad et al. (1999) Plant Cell 11:407-416; Kinoshita et al. (2001) Proc. Natl. Acad. Sci. USA 98(24):14156-14161). These results suggest FIE protein encoded by a single-copy gene in the Arabidopsis genome may form distinct complexes in different plant tissues and participate in repression of several developmental programs.

As has been shown by RT-PCR, the ZmFIE1 gene is active only in kernels after pollination, but ZmFIE2 has very broad expression in virtually all tissues, much like the Arabidopsis FIE. Because both ZmFIE genes are expressed in developing kernels, their expression in this organ have been studied by different methods to understand whether these genes have a functional redundancy. The RNA gel blot experiments revealed significant differences between the transcriptional activity of these two genes. ZmFIE1 RNA revealed the inducible pattern of expression with a maximum activity around 9 DAP. ZmFIE2 RNA is detected at a steady level across the various developmental stages as very low-abundance transcripts. Moreover, the FIE genes are active in different tissues of the developing kernels. ZmFIE1 is active in the endosperm, as shown by the MPSS RNA profiling experiments (FIG. 1). The small number of tag sequences detected in the embryo tissues may be explained by contamination of the embryos with endosperm cells during tissue dissection, particularly in view of the sensitivity of detection in MPSS experiments (1 molecule per million). ZmFIE2 cDNA is not suitable for MPSS analysis, as it lacks the restriction site for enzyme Dpnll, which is used to generate tags. But, in situ hybridization experiments have shown that the ZmFIE2 transcripts occur in the embryo, not in the endosperm, suggesting that these two ZmFIE genes are active in different tissues of the developing kernels. Thus the expression patterns argue in favor of the nonredundant function of these two FIE proteins in developing kernels.

Of importance is the pattern of ZmFIE expression in the female gametophyte, i.e., the embryo sac before fertilization. The Arabidopsis FIE mRNA is found before fertilization in the embryo sac (Luo et al. (2000) Proc. Natl. Acad. Sci. USA 97(19):10637-10642), confirming its function as a repressor of endosperm development. Expression of ZmFIE is different in the female gametophyte as well. ZmFIE1 mRNA is not detected in ovules by RNA blot analysis or by MPSS profiling (FIG. 1). The high sensitivity of MPSS provides strong evidence of no basal expression, or low expression, of ZmFIE1 in ovules before pollination. Conversely the in situ hybridization data show a detectable amount of ZmFIE2 RNA in the embryo sac. Out of these two maize FIE proteins, only FIE2 is a candidate for a repressor of endosperm development before fertilization, the function performed by the Arabidopsis FIE protein. Loss-of-function mutant analysis will confirm this function.

ZmFIE Genes Are Regulated by Imprinting.

The prominent feature of the Arabidopsis FIS genes is their parent-of-origin effect in developing seeds (Grossniklaus et al. (1998) Science 280(5362):446-450; Ohad et al. (1996) Proc. Natl. Acad. Sci. USA 93(11):5319-5324; Luo et al. (2000) Proc. Natl. Acad. Sci. USA 97(19):10637-10642). The wild-type paternal alleles do not rescue the maternally derived mutant alleles (Grossniklaus et al.(1998) Science 280(5362):446-450; Ohad et al. (1996) Proc. Natl. Acad. Sci. USA 93(11):5319-5324); and paternally derived allele expression is delayed (FIE and MEA) or nonexistent (FIS2) (Luo et al. (2000) Proc. Natl. Acad. Sci. USA 97(19): 10637-10642). FIS genes are regulated by imprinting, emphasizing the importance of maternal control of early seed development.

To investigate the possibility that ZmFIE genes are also imprinted, several experiments were conducted to monitor the paternal and maternal FIE RNAs in developing kernels. Both genes show silencing of paternal allele expression with a distinct temporal pattern.

The ZmFIE2 paternal allele shows no detectable activity until 10 DAP. This pattern of silencing is very similar to AtFIE in which imprinting is in force until 3 DAP and later breaks down (Luo et al. (2000) Proc. Natl. Acad. Sci. USA 97(19):10637-10642).

The ZmFIE1 paternal allele shows no expression at any developmental stages (FIG. 2), resembling in this aspect the Arabidopsis gene FIS2 (Luo et al. (2000) Proc. Natl. Acad. Sci. USA 97(19): 10637-10642). ZmFIE and AtFIS2 are different types of proteins, but they are encoded by genes with very specific patterns of expression in the endosperm. FIS2::GUS activity was observed only in endosperm of the developing seed (Luo et al. (2000) Proc. Natl. Acad. Sci. USA 97(19):10637-10642). ZmFIE1 expression is also limited to the endosperm. This suggests that genes that are expressed only in endosperm, similar to AtFIS2 and ZmFIE1, undergo more stringent, permanent imprinting. Genes that are expressed in both embryo and endosperm, like AtFIE and MEA, are regulated by less stringent, temporal imprinting, which causes a delay in expression of paternal alleles, and subsequent breakdown of imprinting later in development. The ZmFIE2 gene belongs to this group, which is regulated by a temporal type of imprinting.

Mammalian Models of Imprinting May Be Applicable to Plants.

ZmFIE genes have a differential parent-of-origin activity and are regulated by permanent and temporal types of imprinting. The presence of repeated sequences is a common feature of epigenetically silenced and imprinted genes (Alleman and Doctor (2000) Plant Mol. Biol. 43:147-161). Fragments of 12 kb of the Mo17 genomic loci of ZmFIE have been sequenced (FIG. 4). A complex repetitive structure is found 5′ upstream of the ZmFIE2 coding region. Repeats occupy the 2.6 kb fragment adjacent to a putative promoter and a 1 kb fragment further upstream. The entire 6 kb upstream fragment does not share any homology to transposable elements, which are abundant sequences of the intergenic regions in the maize genome. It appears that the structural repetitive complex upstream of the ZmFIE2 gene is an integral part of this gene and may be a cis-element regulating ZmFIE2 activity. A critical aspect of the ZmFIE2 expression is the delayed activity of the paternal allele in the developing kernels, referenced herein as temporal imprinting. The upstream-positioned repeats may be involved in setting imprinting marks on the ZmFIE2 gene during gametogenesis. It is possible that specific proteins that function as activators or repressors of gene expression bind with these repeats. These complexes might be temporally associated with the upstream sequence but degraded during kernel development.

The genomic sequence of the ZmFIE1 gene does not possess such obvious structures as repeats. Moreover, the promoter region of ZmFIE1 is relatively short, approximately 780 nt between the putative RNA start and the LTR of a retrotransposon RIRE (FIG. 4). The special feature of the ZmFie1 gene is the 290 bp intron positioned at the 5′ untranslated region. The first exon and intron are often required for high level expression of the reporter that may be a result of the increased level or stability of the mature cytoplasmic mRNA constructs (Kim and Guiltinan (1999) Plant Physiol. 121(1):225-236); Clancy et al. (1994)). It is very likely that the 5′ UTR intron of ZmFIE1 plays a regulatory role or determines the tissue specificity of FIE1 protein expression. There are no indications in the literature that introns are involved in genomic imprinting. It has been proposed that CpG islands might be common imprinting elements in mammalian genes regulated by imprinting (Wutz et al. (1997) Nature 389(6652):745-749). Methylation of these islands during gametogenesis create the imprinting signals that maintain expression of the maternal or paternal alleles. The comparative analysis of mouse and human imprinted domains suggests a two-island rule for imprinted genes (Onyango et al. (2000) Genome Res. 10(11):1697-1710). Imprinted genes show two or more conserved CpG islands upstream or with the gene, while non-imprinted genes are associated with at most one CpG island. CpG islands are normally unmethylated and associated with actively transcribed genes, but allele-specific methylation of CpG islands appears to mark imprinted genes in mammals (Wutz et al. (1997) Nature 389(6652):745-749).

The distribution of CpG islands within the ZmFie1 and ZmFie2 genomic sequences was searched using a definition of CpG islands as sequences of >200 bp with a GC content >0.5 and an observed-to-expected CpG dinucleotide content >0.6. This analysis revealed two CpG islands in ZmFIE1 and one CpG in ZmFIE2 (FIG. 7). The results concur with a two-island rule. The ZmFIE1 gene, in which the paternal allele is silenced during all stages of kernel development, shows two CpG islands. The ZmFIE2 gene, which demonstrates a more relaxed type of imprinting, shows only one CpG island, implying a different mechanism of delayed expression of the paternal allele, which is not associated with DNA methylation. The data presented herein suggest that CpG islands may be the imprint marks in plants as well.

This assumption generates several predictions that may be experimentally tested. Transgenic constructs with a reporter gene placed between CpG islands should mimic the parent-of-origin pattern of expression of the ZmFIE1 gene. A pattern of DNA methylation across the ZmFIE1 gene can be tested in DNAs isolated from the male and female gametophytic tissues (pollen and ovules), and endosperm. This would provide evidence for differential methylation of the islands during gametogenesis and its maintenance during endosperm development. Further, imprinted antisense transcripts are observed in all major imprinting models in mammals (Fu et al. (2000) Proc. Natl. Acad. Sci. USA 99(2):1082-1087), which were proposed originally as the sense/antisense competition model for preferential allelic expression of the mouse Igf2r gene (Wutz et al. (1997) Nature 389(6652):745-749).

The two-island rule can be used to predict imprinted genes in plants. In this manner, a search of 2,000 full-length transcripts of annotated genes reveals that 10% of them fall within the category of two and more CpG islands. Relatively few genes are described in plants as being regulated by imprinting, but this approach provides a potentially useful predictive tool for identification of imprinted genes. Support for the relevance of this approach comes from the finding of the α tubulin cDNA (tubα4), which shows two CpG islands. Imprinting of the maize α tubulin genes (families tubα3 and tubα4) has been documented (Lund et al. (1995) Mol. Gen. Genet. 246(6):716-722). Moreover, expression of the sense and antisense transcripts of the α tubulin genes were demonstrated earlier (Dolfini et al. (1993) Mol. Gen. Genet. 241(1-2):161-169). Having demonstrated the applicability of the two CpG island rule for imprinting in the maize FIE genes, it seems probable that this rule operates generally in plants, and suggests that the general mechanism of imprinting may be conserved in evolution across the kingdoms.

Two FIE Genes Reflects the Maize Genome Evolution.

The ZmFIE genes are located in the regions of chromosome 4 and chromosome 10, which are very likely duplicated in the maize genome (Helentjaris (1995) Maize Newsletter 69:67-81; Gaut and Doebley (1997) Proc. Natl. Acad. Sci. USA 94(13):6809-6814). The phylogenetic analysis of the known plant FIE proteins shows that sorghum and the maize FIE2 protein are more closely related to each other than to the maize FIE1 protein (FIG. 8). This observation concurs with the hypothesis that the maize genome is a product of a segmental allotetraploid event (Gaut and Doebley (1997) Proc. Natl. Acad. Sci. USA 94(13):6809-6814). These authors provided evidence that “at least some elements of the sorghum genome share a more recent ancestor with one of the two maize subgenomes than the two maize subgenomes share to each other” (Gaut and Doebley (1997) Proc. Natl. Acad. Sci. USA 94(13):6809-6814). One can speculate that a segmental duplication of chromosome 10 around a centromeric region (Bin 10.03) has its origin from the sorghum-related progenitor. The orthologous region on chromosome 4 around the centromeric region (bin 4.05) carrying the ZmFIE1 gene might originate from the second ancient genome that was more diverged from sorghum.

Despite the similarity between ZmFIE1 and ZmFIE2 genes, they are differently regulated. The ZmFIE2 gene has a broad expression pattern whereas ZmFIE1 expression appears to be restricted to developing kernels. These genes are regulated by different types of imprinting. The data herein strongly support the nonredundant function of these genes. ZmFIE2 gene is very likely to be a functional homologue of the Arabidopsis FIE genes with multiple functions during maize development, such as preventing endosperm development before fertilization, and may be involved in functions for embryo growth and control of flowering. The second maize gene, ZmFIE1, has evolved for a kernel-specific function, most likely in endosperm development. Experiments with null mutant analysis will further elucidate the function of these genes in maize.

### Example 2

### Imprinting of the Maize Endosperm-Specific Gene FIE1 Is Mediated by Demethylation of the Maternal Complements

Significant progress has been made on revealing imprinting mechanisms in mammals, but no such progress has been made in plants. The underlying mechanism of mammalian imprinting is differential DNA methylation of maternal versus paternal alleles, a process that takes place during gametogenesis (Constancia M, et. al., Genome Res. 1998, 8:881-900). DNA methylation means the occurrence of 5-methylcytosine instead of cytosine in the context of CpG sequence. The major function of cytosine methylation is transcriptional repression.

Most of the CpG sites in higher eukaryotes are methylated with the exception of CpG islands, which are stretches of DNA enriched in CG di-nucleotides (Ponger et. al., 2001, Genome Res 11:1854-1860). Imprinted mammalian genes show differential DNA methylation in CpG islands (Reik, et al.,2001, Nat Rev Genet 2, 21-32). Onyango et al. (Genome Res, 2000, 10:1697-1710) reported that the mammalian imprinted genes show two or more CpG islands within gene sequences, an observation referred to as the two-island rule. As shown herein, the maize FIE1 gene is imprinted and contains two CpG islands in its genomic sequence. This suggests some similarity between imprinting mechanisms in plants and mammals. The role of cytosine methylation in imprinting of the ZmFIE1 gene was further investigated, as follows.

Results

DNA methylation assay of ZmFIE genes in leaves, embryos and endosperms. To investigate whether cytosine methylation occurs within ZmFIE genes and correlates with imprinting, a quick and simple method was developed; it comprises DNA digestion with methylation-sensitive restriction enzymes, followed by PCR amplification across the restriction sites. PCR amplification of digested DNA occurs only if the cytosines were methylated and thus protected the DNA from digestion.

Commonly used enzymes Hpall and Mspl were chosen for this analysis, but any other methylation-sensitive enzymes or mixture of several enzymes could be used. Both enzymes recognize CCGG sites, but show different sensitivities to cytosine methylation (New England Biolab catalog). Hpall does not cut DNA if either cytosine is methylated. Mspl cuts DNA with the internal cytosine methylated, but does not cut DNA when the external cytosine is methylated.

PCR primers positioned across the restriction CCGG sites will amplify the Hpall/Mspl digested DNA if CCGG sites are methylated. PCR reaction on unmethylated Hpall/Mspl digested DNA will fail.

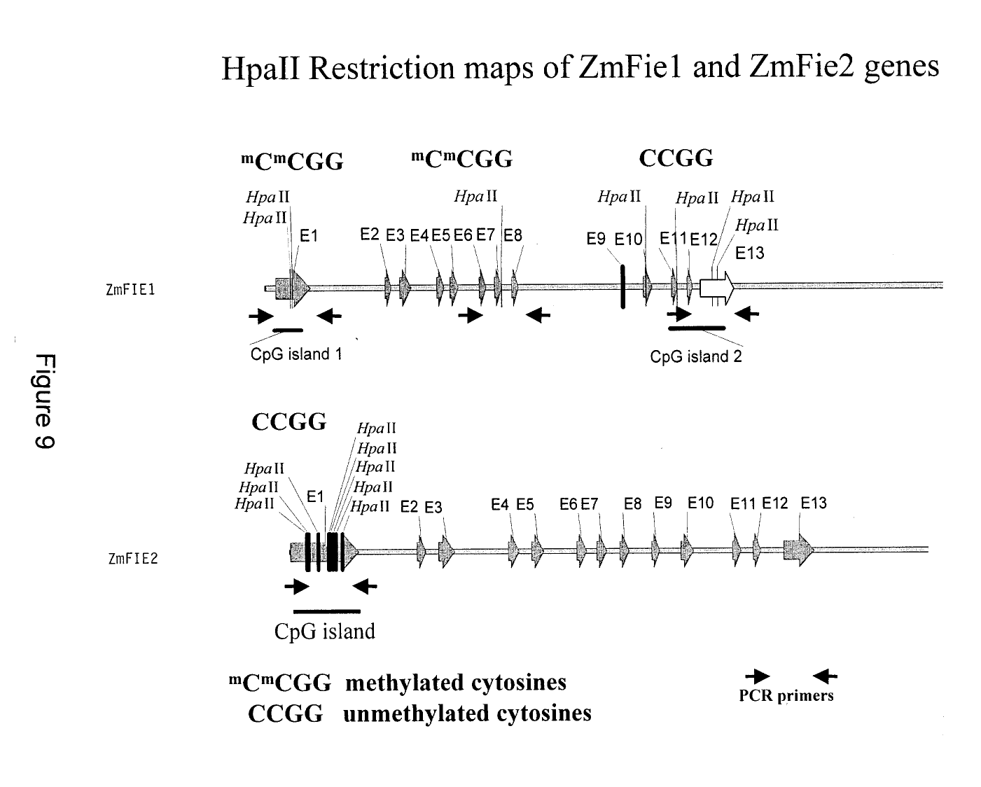

The restriction maps of ZmFie1 and ZmFie2 genomic sequences (FIG. 9) show a distinct distribution of Hpall/Mspl sites (CCGG) across the genes, scattered along ZmFIE1 and grouped in a cluster in ZmFIE2.

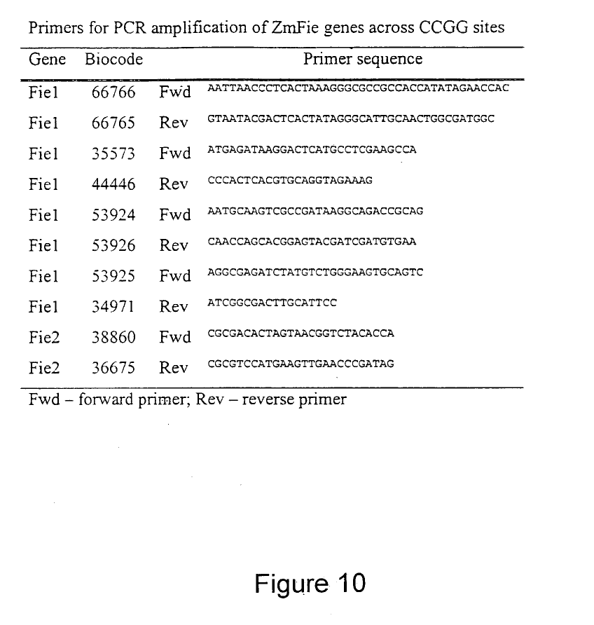

As shown previously, the ZmFIE1 gene has two GC-rich segments defined as CpG islands. The first island is located within exon 1. The second island covers exons 11-12 and 3′UTR. The islands have two and three Hpall sites, respectively. There are also Hpall sites in exon 7 and exon 10. Four pairs of primers were designed around clusters of Hpall sites to monitor cytosine methylation in CCGG sites (FIG. 10).

The ZmFIE2 gene has one CpG island within exon 1. Eight Hpall sites are grouped there. No Hpall sites are present in any other segments of the ZmFIE2 gene. One pair of primers was designed for the ZmFIE2 gene (FIG. 10).

DNA samples isolated from embryos and endosperms of 14DAP kernels of reciprocal crosses between public inbred lines B73 and Mo17 were digested with Hpall and Mspl enzymes separately. DNA extracted from leaves of B73 inbred was used as a control. PCR amplification of an equal amount of undigested and digested DNA was performed and PCR products were visualized on agarose gels.

For the ZmFIE2 gene, none of the digested DNAs support PCR amplification, indicating that CCGG sites within ZmFIE2 are unmethylated in tissues tested (leaves, embryos, endosperms). These results are in good agreement with the expression pattern of the ZmFIE2 gene. As shown previously, this gene is expressed in all tissues throughout development. The unmethylated status of the gene is consistent with its transcriptional activity.

Conversely, a specific pattern of cytosine methylation across the ZmFIE1 gene was found. CCGG sites within CpG island 1 and exon 7 are methylated in both cytosines because Hpall and Mspl digested DNAs are amplified effectively. This pattern of cytosine methylation is present in all tissues tested (leaves, embryos, endosperms). But CpG island 2, which is located in the downstream portion of the gene, is methylated very weakly in embryo and leaf DNA, and is barely detectable by PCR in the endosperm. Results clearly demonstrate that there is a gradient of cytosine methylation along the ZmFIE1 gene, being heavily methylated at the 5′ end and unmethylated at the 3′ end of the gene. DNA methylation of the ZmFIE1 gene correlates well with a repressed status of this gene in all maize tissues except the endosperm. As was shown previously, only maternally transmitted ZmFie1 allele is expressed in the endosperm; maternally transmitted ZmFie1 allele must be demethylated in the endosperm DNA.

Maternally derived fie1 alleles are demethylated in the endosperm

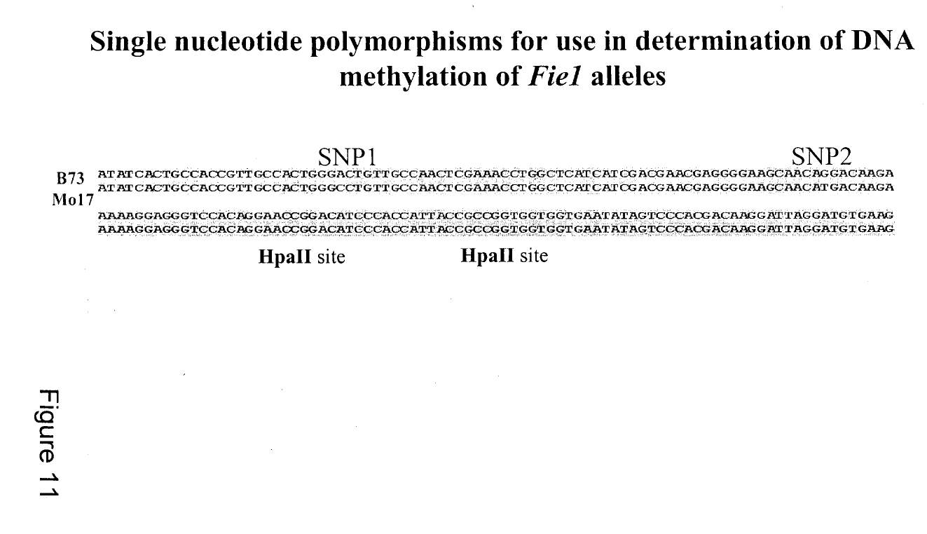

Status of cytosine methylation of the maternally- and paternally-transmitted ZmFIE1 alleles in the endosperm DNA was determined by means of two SNPs (single nucleotide polymorphism) present in exon 1 of B73 and Mo17 inbred lines (FIG. 11). PCR primers were designed around the SNPs and Hpall sites. If both alleles were methylated at ^{m}C^{m}CGG sites, the sequences of PCR products would show traces of both SNPs. If only one allele were methylated at ^{m}C^{m}CGG sites, the sequence of PCR products would have SNPs from only one parent.

To facilitate direct sequencing of PCR products, ZmFIE1gene-specific primers were extended with T3 and T7 primers at 5′ ends. DNA isolated from embryos and endosperms of the B73 and Mo17 reciprocal crosses was digested to completion with Hpall and Mspl enzymes. The digested DNA was amplified by PCR, and the fragments were sequenced with T3 and T7 primers. Chromatograms of the nucleotide traces of PCR products from embryo DNAs showed a mixture of SNPs from both parents, B73 and Mo17. This is strong evidence that both parental alleles are methylated in the embryo. Conversely, the chromatograms of PCR products generated from the endosperm DNA show SNPs from the paternally transmitted alleles and complete absence of traces from the maternally transmitted alleles. Undigested DNAs, used as a control, showed a mixture of traces from both parents.

Discussion

This indicates that the ZmFie1 paternal allele remains methylated in the endosperm, but the maternal allele undergoes de-methylation followed by transcriptional activation. Data suggest that the methylated state is a default for the FIE1 gene; thus transcriptional activation of the maternal fie1 complements is achieved through demethylation. The paternal allele remains methylated and transcriptionally inactive during endosperm development. Maternal-specific demethylation explains the mechanism of imprinting of the ZmFIE1 gene. It is very likely that demethylation of the maternal genes is taking place in the central cell of the female gametophytes before fertilization.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the invention.

We claim:

1. A method of identifying imprinted genes in a plant, comprising identification of two or more CpG islands located partially or completely within the coding region.

2. The method of claim 1 wherein said plant is of the species Zea mays.

3. A method of identifying plant genes involved in endosperm development, comprising identification of two or more CpG islands located partially or completely within the coding region of said genes.

4. A method of silencing paternally-transmitted alleles of a plant gene, comprising transformation of a plant with a construct comprising at least two CpG islands operably linked to the coding sequence of the gene of interest and a promoter that drives expression in plants.

5. A method of detecting cytosine methylation in a polynucleotide of interest, comprising:
(a) Restriction of said polynucleotide with methylation-sensitive restriction enzymes, followed by
(b) PCR amplification using primers positioned across the restriction sites for the methylation-sensitive enzymes wherein PCR amplification of digested DNA will occur only where methylation protects the polynucleotide from restriction.

6. A method of controlling plant gene expression in the endosperm, comprising demethylation of CpG islands in the allele contributed by the female parent.

7. The method of claim 6, wherein the plant is of the species Zea mays.


SEQUENCE LISTING (6 sequences)

SEQ ID NO: 1 - DNA, 17 bp, Zea mays
gatctagtgtgtggctg

SEQ ID NO: 2 - DNA, 24 bp, Zea mays
cgtgaaggcaaaatctacgtgtgg

SEQ ID NO: 3 - DNA, 29 bp, Zea mays
cattacgttacaaatatgtgaaccaaacg

SEQ ID NO: 4 - DNA, 31 bp, Zea mays
cagaacaaacagatgacaacggttcccaaag

SEQ ID NO: 5 - DNA, 13031 bp, Zea mays
  unsure (11384)...(11481): N = A, T, C or G
ccgatcattcgtttgttcgatcatttgatcgttcatcgttcgttcatagttcctattcatcgttcatcgtttgttcatagtacttattcatcgttcatcgttcgttcatagttcctattcatcgttcatcgttactattcatcgacactattcaccatcgttactattcattgttactatttaccggctctattcgtcatcgttactattcatcgttgctatttatggtagctttttcgttgttactattcatcgatcatccgatcgccccaaatttcaactactcatccatcatgttgtccagtccacctaagaccagccagacccatattccagtcatacgaactcctgtgattgtgattttccttccagtagggaacctcccatctggtcacccatcctaggtttctccaagttgagcatgcttaactttgagattcctttgaaccaggcttccaaactcagattccaataattcttgtttctaaattcttatcaaactattccctatccaaccatgtcatcccttaagcctggtccatattccagaaaactcccaaaatactcttgtcccatattctgcatataactctcctgttcatactaagtcagacgattcattcgtcactattctcaccaacagtgaacttcactgtgctacaccacatacactcagctataaatacacccagctaccctctccctctccacacacactcaacaccctcagccaaggcaaacacctcacccactcagttactccgctctaccggctacacgcatagtgtcgcttcgcctccagtccaccctcctggtaagcacctccgctccaccaccagtaatatcacaacaccacatgacacagattctactcaagactctacccatccatatatcgctattctgaccactatactaaatatttgttggtatacttgctggtttgtatgtttgcttgttcatgttgcatagttatcggagcgttcgtgccatcacgtggaggccagatctgcaagtctacgccaggcggtggagccagaagccagttccgcgagctctccttcccccttcactggataagcacagcaagctcactggatccctttgatgcataaattacctatgatttttcaaccacaaccctcagcctgttattttatgcataatatgattttgagacaagttattatggccacccagccgcttgtcgcaatcaatccttgatatatttgttacaaatgatttgagaaaaggtgtgagttttcaaaagaaaatgcttttcaaaatgtgtatgatgaagggttttcacccttatcaccttttaatagggatgatcaaggactccctggtttaggggagggcctaaggtgatggctcagctggtttaggtgtgagcagaaggattgtcccctcacataaggaccgatttgtcatccgtcactacctgtactcatgataagtacaaccactcgagactgtatgggcaatcactcaatctgaactcgtacggtccaaccctagggttatgaaggctggggagcaccgggaggataaggagggagaatgttttgtccggtttggacatggcggtggcctgactccttccggtataaccgttaaggtaaggacgtgcgaggaaagaaagagatccggcattcgggcctcacgacggtgagatcgcagaaaccagactagtgggtaaagtgtacccctctgcgcagagtttgaaaacctattcgaatagtctgtgtccacaggaatggacgagtctggtgtggtatgacaattagtgttttgttttcaaaaaagaatgtgcgtttgagaaaagtggtttttaaaaggtccggcggttgagccgtgagctatggtggacgggaagtccagtagctgtttttgaaaacgaaaaccagtgggaaactgctgagatacctggatggtttagtccaggggattttgttctaatattgaaaaaaaattcttgctcctttgggagaggatgcgctttgcaaaatacaaaatgttttacaaaataaccctgcataaaatattgttgtttctgcaaaatatcctgagctccacatattccatgcattatatctgatttccccattccgcgggtgatggtgggctgctgagtacgtttgtactcacccttgcttatttgttgtttttcaaaaaaaggagatcgggtaagagttacgactgttcccaaccttgcctgtggttgttggaccgctgatttgcttcgctgcgtatatcgggctgcttcatccccactctgatgatatgtcccaagttgtggaccaactcttaaagttgatcgccacctttataggtttgtctcgtttaagcagatctggaatcatttgatgtataaatgtgtttactagcctcctgggactagtaattgtatcacatttgagtcctagaggatcgggacgcttcaatgatcaatgggtggatcacaatagtcggttataatggctatatcaacagttataatcacattaaatgtgtcatcagatgttagataaagtctgtcgtggatgatctgtttgtgcttctcgacggtccatgagtgacgctaaaattcattttaccaaacctagcaccttcgagttggtctgatcttgaatagtcagacggttcacgactgaggttgaacgatccacgcaaggtgttggacgatactttctttttctttggatgctccgtagtagatgtgtcggttttgacatagttcctgtccgaactccatacagtccatagtagatgtgtcggttttggtactctagacggcccgagtcaggggtctggacagtcctggacttgctgagttgaggtttgatctttctttagttatttcttacatacctatgttcatacacttagcaaactagttagcttcaccaaaacaagtgtggaaaaaggtttttaggccaatttccctttcacctttataactacctagttacaaagtagagtttgatagtccctaagtatgtcaattcacatcttgagtacatgcgacaatctcatgtctaaggatacatggtacaggttgcaagaagaaaattgtcacaatatctcatgttgggtcagtacagactcatgtcatacatgcacccatattattagttttacatctccatgtccatgacttacgaaacatagtcatcaactaatacatatgatagtcattgactctaactagggacatcttctagaacaaccatacaagaaaagagtctcacaaacaattcacataattgctaatcaatacaaggtgtccttcacagatattcaattaaacaatatatcatggatgcaacawaatatgctcatctctatgattatctctagggcatatttctaacacaatgacatgtctaagtgtagtatgtcaaaacatggatagtaatatagatggtaagaggtcatttttattaatataattaacaaagatagatagggtgaccaattttgtaaaagcaccattcatagacttttagtgggaggtggatgctctacccgcctccgtaaagccaaagtggttgcatgcaaattgytaggatatagtaatgcaaggaaccaagctaaggcatgtaagtgaaacccaaacaagaagttaagaagcttccaaaatgaacaaagtacaagaatgaagctaaaagagaaactttcagccttctccaatctccagcaagatcccttcgatagatggtatctaattttttcctactatgaaaacctatatcacctagtagaatagaggacaaagcttacgcctactatatatatccaatatgtatagttagatactaagttcttttttctcttctcttcattcacttttcaactaggtttggaattaagtttttggattggcatagacaatggcatggttgtataggtgttcttaaccatcacagttatgagtttgacttgttttttatattcaagttacaaggtcattttgtgctagccacagcctagcaatcgaggggctacacatgtggattaaggacaaggcccaacccatgtacgatccaaggacacccttgtaatttttatactcatcaaggattagggggaaataactcccttctatataaaggtctttccactttgcttctcactctcccttattaggttaaacacaaaatgtgcatcgccgccgccaccatatagaaccacttatcacgaaccgccgccatcacatccactgcctcaactagtgttaccacctatggttcattgttgtgtctgcttcttgtagcactgttggtctacaaacattcatatttctctcaacatctggcacaggtaagcccataagccctaaccctagatctccatatttagttatttcagttcttgatgagcaaatatgaaactaaattagtttgctaataagaaatttaactacttttcctcttgaagacctcctatccctatatgaacccacatccaaaacccctctagcaaagtgtggctagctttcccatgccatgaaccttcaacaatgatagtatcagtaatgcacttccataaaagggttcatatttaattttagtttttctttttggtgttttaattaagctttgagacttgatttgaagtattaaataaacccttcaaatttctttctaactttgataatacactattcaatgacaatgcacttccttaaatccctatacttcacagcatgccgccttccaaagcacgccgaaagaggtcacttcgtgatatcactgccaccgttgccactgggcctgttgccaactcgaaacctggctcatcatcgacgaacgaggggaagcaacatgacaagaaaaaggagggtccacaggaaccggacatcccaccattaccgccggtggtggtgaatatagtcccacgacaaggattaggatgtgaagtagtggaagggctactcgtgcctagtcggaagcgagagtacaagcccaatagcaagtatactgtgggaaatcacccgatctatgccatcgggttcaatttcattgacatgcgctactatgatgtctttgccatcgccagttgcaatagtgtaagcaaccgacttctccctacctcttgtttgctatccttttatcctattgaggtttggggagttctatatggtgaacgaaaatggaagttatgattttggtgggattggatcttggtttataactagaaaaggatttgagtacaggttatgatgtgtggctttatggtagggaaacttaatatcttttcctattttgttttttggcatcacgagtaatggtttgggaaataaaagggaaaatgatttaaaattatttctcaatagagcatgcccttttacatagggacattttagtcattttacacacactttagtcattttacacaccgtaattatgtcacaatcaaagaatcattccttggttcaattgaatgagatgattcaactagttcacatctctatacctaacaatatagtttttcataactaaagctttgagacttgatttgaagtattaaataaacccttcaaatttctttctaactttgataatacactattcaatgacaatgcacttccttaaatccctatacttcacagcatgccgccttccaaagcacgccgaaagaggtcacttcgtgatatcactgccaccgttgccactgggcctgttgccaactcgaaacctggctcatcatcgacgaacgaggggaagcaacatgacaagaaaaaggagggtccacaggaaccggacatcccaccattaccgccggtggtggtgaatatagtcccacgacaaggattaggatgtgaagtagtggaagggctactcgtgcctagtcggaagcgagagtacaagcccaatagcaagtatactgtgggaaatcacccgatctatgccatcgggttcaatttcattgacatgcgctactatgatgtctttgccatcgccagttgcaatagtgtaagcaaccgacttctccctacctcttgtttgctatccatttatcctattgaggtttggggagttctatatggtgaacgaaaatggaagttatgattttggtgggattggatcttggtttataactagaaaaggatttgagtacaggttatgatgtgtggctttatggtagggaaacttaatatcttttcctattttgttttttggcatcacgagtaatggtttgggaaataaaagggaaaatgatttaaaattatttctcaatagagcatgcccttttacatagggacattttagtcattttacacacactttagtcattttacacaccgtaattatgtcacaatcaaagaatcattccttggttcaattgaatgagatgattcaactagttcacatctctatacctaacaatatagtttttcataactagaattcttaaaaagaattaatatgaacctaaatattatttcactttcttgccccttataatataatacatttgtcactcccattttggcaagggtggtgggtattttgggggatggaatgttactatttttaatttgattagaagctataagctttggctatatttttattaggaatttgatgttcattttcaatatattgtgatctattttcttaaaatgtgaatttgttgtgtattttgattagttcgatgaagagtgtttataagatatgatttttaaattctcttacgacgaaacaatattatgttactttcatctattcatcttgaggaatcacctacctcacttcttgatcttgcaggtgataatttaccgatgccttgagaatggtggttttggtcttctacaaaattatgttgatgaggatgtgagaaagacaatgcctggtgcatgtggttgttaatgttaatttgataatatgcttttatctaatgtctgtggtgcctatttatctcagaaggatgagtcattctacactctaagctggaccatcgatcaagttgatagctcaccgctgttggtggccgctggaagcaatcggatcattcgggtcatcaattgtgctaccgaaaagttagataaggtccctgcccctgtgcttactctatgtttgtatggaaaagttgattgaacgttgatgttcacatatcaatatttcagtagtttagttgaaatacaatttatttatgctctctattcttgaacatcagttgactttgctttgattaagcaatggtcttgctcatacaatattctaggagttgaatattcaatatgcctgttacatgatagcaaatacatagtgaactaggacatgtactaaatatttaatttccctttatgacattctctagagcttagttggccatggtggttcaatacatgagataaggactcatgcctcgaagccatcactcatcatttctgccagcaaggttagtaataaatttgtcgtgtgtcgatttttttacactttttaacatgacattattctataggatgaatctattaggctatggaatgtccatactgggatttgcatcttagtctttgcaggggctggaggccatcgacatgatgtgttgagtgttgtaagtatcgattgcatcttgtctagacattgttttaaatatcacttgccccgaagataacactcattagaattctaatgttaccatttgttattgagcatgccaaatttcaattttaacatcatagataaaataagaccccacaattacttttactgtttatctacttccattacattaggcataaagttactgataaaaaagacaatcttttatctgaaggacttccaccctaccgaggttgggatttttgcaagttgtggcatggacaatactgtgaagatttggtcaatgaaaggtttgggaactactttaaactagcttcatgtttacattttgtgttgtatgttgcatatcatcgacaaatattgccaatgttgtcacagaattttggatatatgttgaaaaatcatattcatggactggccatccatcaaagtttccaacgaggaatatccagtttccggtatgttaagtagctataatcacctgagctcctttctttttttgcaaactattgttggtgttcagttttcatgccattcaagcatacatgtttcttttcttttaggtcttgactgctgcagtacactctgactatgttgattgtaccaagatggcttggtgacttcatcctatcaaaaggtaaattcttcatttgttaaatggctatacatttttttataaaggaaattttttattaatttcaagcactttagattgaaataatacaaaatcttaaaaaacatttttggcctccatttaaacaagcacaaatccaacaaaaatgagtaaaccaacccattctagtgaatattaatgcataaactagattgctacccatatgtctagaaaaagtagccttgaccgcgtatcttaattgtcaccatgccgccacaaccaaaccgtgcaaatatggtttttggagaatggaccaagtaagaaaccaatcaataattgagtatatagcatgcacaggagaaatagatctcttattttcaagaacaatggtattttttattaaccataggaccaacaagtagcgactacccatagcaaaactaatggcttcagattattactggttgttgaagtgtatacgtggtttgcctactttctcccaatagtttaagcttttggattgaatcgattagtgcgttcactcttacatggtatcaaagttagcaattttgggtttgaatcctaacggaagctttatttgtgacttcacctcttgttttccatttcctttctacctgcacgtgagtgggggtgttgaagtgtataagtggattgcctaccttatcaaccttttggattaaactggttattggttagtgtgttcactcctacacctaagtatgaggtttagttatccagtagccaattagattatgcacagtggacacttcacatgtgcaactagcactcaaaacataagtctttaattgtctcatcttatgacaaaacaacatatttcactaccattctataacatcttgatttgtacatcagtcttgttaatgctaaatagtgagatttgatcgtcaattggccagttggatgtaaattccagtgaaatacatcttgaccttgggttaaatggacattagcaatgtgtgggaacaaattgttggtttgggtacaccaaactgttggtttttaattagtagattagtttgtaacacatttccttttatcagtgttagtattggtttattatgcatagggaaggatctgatatgtgataattaacatggatttgcagagtgtaaagaatgcagttttgctttgggaaccaaaaccagacaagcgtaggcctggggaggtgacacgctttaccttctcgtcccgaattctgcacctatttttatattactatcatactcatctacagtttaaaacttgtcccgcaatcttttcagtttctgagcactaaatttatacctctgaatcagtatagtcgttttctctttgttcgtataggggagtgttgatgttcttcagaagtacccggtgccaaagtgttcattatggtttatgaaattttcatgtgatttttactccaaccagatggcaataggtaatgcctttaattttgtgaagactgttttggcactaaagctttacgtacgtaatattagttttatatcttgtacattgatggaaaatagattgctcaatatctatatatatgactatatcttgggttagattctaaggaacaaactctcccagagtacggttctgaataacaaccatctgctgctgctgcttaatgcgaacaggcaacaataaaggcgagatctatgtctgggaagtgcagtccagcccgcccgtcttaattgaccggtaaatttccagttcttctcctcctcgcatcggttcctgcatgggtagctagctagtaactccgacgcttctgctggatgcaaacacttgtgcattttcaggctgtgcaaccaggaatgcaagtcgccgataaggcagaccgcagtgtcattcgacggaaggcacgtacgcactacgactctcactatctgctcatgcatgcattcaccgcacgtacgtgtgatgtgctcgctcgcttcctccttttgtgatggtgtctctctcacttgcccagcacgatcttggagccgccgacgacggcggatctggcgcggtgggacgaagtggaccctgctgcttccagctccaaacctgatcaagctgctgcgcccgccgccggtgcgggtgccgacgccgacgccgacgcctgagcgagaggaccgtcgtcgcccgccggttcacatcgatcgtactccgtgctggctgattacctttacccattgggatgttttggttcagagtcgccagatctagtgtgtggctgaacgttgaatgttaggatgctgctgcttgttatgctctgagtcttgagttctctttgttaatttgcaccgtggatgagatgaataacttgacgttgcaactttgcatcccatatatgccgtaaatctgccgtctgttgtttgttctgcgttgtctagaattagtggagatgtgctggatacaatgtatgctagtctattaaaccgtgctccactctgagataatcgaccaacttgtcttattattgaaagaactgtggaaaaaaccaaaaaaagtcgttgtggttttgtttattatcaaatatattttacataagacttaaaagttttcattttttcatgaattttttgaataaaccgagtagtcaaagctagggtcaaaaaggcaaacatattatattttaaaatggagagagagtacattgttttaagacgaattgtttaatacaactcgagaatattctgatacattaatcctatgatattaccataaaaaacattaatcctatgatagagtgtataattacaaatgcacaaaggttcttttcatgtgaaatcgtattatagataggggtcatagcgcgcccttgtccctacaacttacgatgttcatgagttaggttagaaaaaggttagagcaagtatactaaagtgacatatgcaggctacaaggaatgccacatcagatttttggtgacgttgaaggaagaaaaatagagggagaaaaaagcgaaccaattgcgaaggtgccttcttccaagggcacggtccatggagtgtggtagccgacatcaaggtagaggattatggtaaagttatttgagcaagtgtctgacaactagcatgaaggcttaggattttctaaatgcatctttgagcgctattgatgtagatgttaatgatttttagggctgatgaccaaaccaaagatgaacatgggaacgnaaggaaggttactgaaagtgtataggcccctagtttagtcttcagtgactaatgataatatatattattgtgactaacaagtgttttatagaaacanggaaagttagatcacaataatagatatgatcaggattattatgtggtacccatcccttattgatgaaaatcaatggttggttctcataggataatcgaaaaggttaaggatcaactgtaaatggagttgttggacacttagagtagtgatttgaccttttttctttggtagtactataaacggacatgaaatgcgtagctttacctaaacaagtctagttaagtatgatgatgcacacttgtgaatactagtgctaggtaaacccatgagatctcatgtgaagttcgaaacaaaacctaattcgaaaagtgattaaaacatgtgacttaacaatgttgtagtagcattggtcgagtttgatgggcacctgatatgggtcactagacatgagtgtgccctgttgtgtttgagtgaagcactagcatatcaggtgtgcaacagatatggtgcacccaggcaggacacccaaagagcttgcaaaattagcctaaaacacttagtgctcaccagacatatctagtgtactactagttattctcgttatatatgaaccctattagttattcttgaattgcttcgatcttttacaaaggaagtagtttttccttcatctccataaactgtggttttccaaaggcattaataataagatttagtatattaaattcaaagttgaggtactttattatcgtgaaaccaacattaatactatagacttaactaaggagtctattggtgcttccttctcatgtattttcttcttgaagtgttccttcatcttggtgctaacgacgacattcaacaatgtgtgctcttacttgattggtttgtatatatggtggtgttcctttacttagtggcaacataccttatcgataactaacccttagtgaaagaaatgaaaatgtacatcccactgggaaatcactcatacccctaagagctaacttaatggaacatcactcatagccctaagggctagttggaagtactttctcatttcctgtataagggctagttcatgattcaacttcttctccatttcttggtgaactatcttagcacgattcctataaaaacatatacaactaaacaaagggtggtggtactgaacacagtggacccaagcactcggaaatgggaaggacaagttgcatggaaaaaacgacaggctgggaactattgtgtcttgtcaagcgtgttcgtccagctataggacatgggtatttatagggcaactagaggttggtatcctaaaatatgtccagacccctagttatcaactacgttcctagataatactgtacaacaaggtaattatagaatagtaagtttgttattctaactccaccccgacaggtgggtccgttgtcgcccggttgagagtgggccctgctcggccaggtcattggcattgtccgtgcagacgtgttcccaatatcgaggcaatgaagttgtttgacacttcttcgggagtcggcgtgaggccttcgcttgctagcgcgaacttgcccacgagcgtcctcaccatgggccccgctgacaagctt

SEQ ID NO: 6 - DNA, 11232 bp, Zea mays
  unsure (11155)...(11232): N = A, T, C, or G
tttttcacaccgttactgtcatctaacagaagcaggtacaaacttgtttttcgttttcaagtcgaattttgaggggcaaaccatagttgcacttccatcgagggacaaaaacacaattgccccttaacttatatagttaaatatagttaacgagcttgctactgagactaacaagtcaaaactattggcttgaccttatattagttttgtcttacactttacaatcgttgatggctgctctagatcttataaacttaagaatattatgactttatcactttatttgtaatggatgtatggatactcattgatgcattatttatggtataaactatagaccatgaatgtatggtgtaatgctatagtatattgttagacttgtgtacatatatattatttatacttaactcacaaacttaatgagtcagctcgaacttataaacgacctgagtcgacctggccttatggcttgttaagataacaagtcaaaccaagccgaactgactcgttatccaaatctacacttacataaacaaaacatgatttcaaattaagattggtacaaaagtgttttgttttattcaattaaaccctacactgtactctttatgtcaacaatagttgatgctacgacaaagcaatgaacattttatggagtagttaattttattgtcctaatgtcaattactattgttagccaaggaatggagtaagccaataaagagtacatatctacgaggaaatttagatatgtgcgtaacttttttaatcgagatacaaaatgtgcaaaataagggtccatgtaacatacatatatttcttgtttttatggtaaaagagtgtataaactataaaggttgttgcttagaagcgggatttaataacatcggttttatattaaccttaagtccctatgcaatacctgtatttttttctaagtacatggtacaaacacaaatacacacatttaagcacacatactcacttgctatgagcacacacacgtaaaccctactcctactagcaccttcaaaagacaaaatagataaatcttgttgacaaagtctattgaaaaatatcaacgtccggtctaaatcttgacaaaatattagcacttgtgccaagttaagaagtgagcacttgaacgtaagtggttagaggaacctaaccaagttagttatgttcaatttttcatgcaagttagcttgctagtttttctatacacaaacattatattagcttataccattgttgggaaattctaactttaatgatttctttgagaaatccataagagcgataaagaggagagagagagagagcaagagatttgtacatgtataaatactatccattttctatttaagaatctagacaaactagcaaatataaatttgaaacataataaagatgggcacctggcatctcctggatattaaaagcgtaccattaaagatatacataattattcacctcttctaggtataaattaccctactaccacattcccctatctctacaaactctctctcattgactcatcaagagagtgccacctctatctctccttctctcttttcaaatgttctacaattatcaaccatcatacaacattcacctttcctaccaaccttgttgatgcttgtctcaactttctctttacctagatcactcatatatatccctatttcaaaggcattaatcatcaaaaacctatagaaaaatcccattatcaaccatgatggagtctgatcgtgagaaacaacagtctcatggcaagaaacaaggtgaccatggtagcaagatgcatgattctgatggcaataaaaatgtgtcagatgaaaagagtcaagagtctggtggtaaggaacacaaatccaatataaagaaacatgaatcacgtagaaagaggtaagacattctccttgaaaatcttggcttcaaactcaagttaaatttatgtacacatgtttatatagagtctagagattttgtgcttaatatatgcatgcacatgagttcaaataatttcataataaaaataaaaaaatcaatatgatcaggaattaaaccatgaaatttttagagacatcatctagattgagttccatggtcataccatgatggttatgtcatttctttccaatataaaaaattccttaacttatactcaaaatgttgattggatggaactttttctatagaattccttgccacatgttgtgtaacaaccatttgtattggtttgcgtctagtccacttttgtgtgttgctattatgtaaataattatttttcaaatccaaagttgttcctccacatatctagaatatattctaattctacaagaatttaaaatgaattgttaacttaagaatgcattgttcaatatatttatgcattttctcccattatgatatatatattctcaatatttggcacataataacttggaacattccttacatttgttgggttgagtgctatatgtttggattcattaattatttacattgatatttttgtagatgtttgtgtttacccaataagaaaaggccattaagaaaataaaatgttattagatagagttagtcttgacatgttatattcttttaataattggattttgtggtatttccaacacattccttccatttaaacctaactccatctctcttatcttcctctatcatataccttatcttctttctacactaacactaatgcttatgtcactcctaaccttgatgcaacctaccaatagtcaattactgttacgttgctagaaccaaagattggtccattggtgcacaatccattagttcctccttcttgggactcttcaaccatcctaactccccaaatgatttcaaaagttttccctaccatgtcatcctactccatatccaatgtctactggtgctagattctatctactgttagcaccaaactaaccacaaaataataatccctacaaatataggtggaggtgatgtaaaattaagggaggggcaattgtaaatggtagtaccatagatatcaaaccttctcaacttagagctatgtctacatagttctagtcctatgaagcatcaaccattttcttactaaactaaatatttttagaggaaggggtggatccttactttcatctccatgagcttccaccccttcctatgagcttatccatcgactgaaagttcctcattgctggagcttacccgttattatcccatgtcatctgacttttgtatgtactattatctttgaagtcgtaggcatgtggtaaattcctaccttaagatccattaatcctccaacacacccttaagacccaaaccataacgcctaaatccaatttcaacatattttaggtgacatgggtatatgtgatattagttacttaatatagcaagctctatcaatgatttttagtcagaaaatggttgatatgtttttagtggttgtactataattgaagaggcacatagagcaagtttttagaccatgaatatatggtgtaaactatagaccatgaatgtatggtgtaatgctatagtatattaattattagacttatggacatatatattatttatacttaactcacaaacttaataagtcagctcgaacttataaaccacctgagtcgaactggccttatggctcgttaagctaataagtcaaaccaagtcgagctgattcattatccaaatctacacttatgtaaacaaaacatgatttcaaattaagattggtacaaaagtgttctgttttattcaattaaacgctacactatactccttatgtcaacaatagttgatgctacgacaaagcaatgaacattttatggattagttaattttattatcctaatgacaattactattgtcagccaaggaatggagtaagccaataaagagtacatatctatgaggaaatttagatatgcgtgcaactttatttttttaatcgagatacagaatgtgcaaaataagggtccatgtaacatacatatatttcttgtttttatggtaaaggagtgtataaactataaaggttgttgcttagaagcgggattttaataacatcaattttatattaaccttaagcccctatccaatacatgtattttatttctaagtacctggtacaagcataaatacacacatttaagcacacatactcacttgttatgagcacacacgtaaaccctactcctactagcaccttcaaaagacaaaacagatagatcttgttgacaaagtctatttatggtataaactatataccatgaatgtatggtgtaatgctatagtatattgttagacttgtgtacatatatattatttatacttaactcacaaacttaataagtcagctcgaacttataaacgacccgagtcgaactggccttatggctcgttaagataacaagtcaaaccaagccgagctgactcattatccaaatctacacttatataaacaaaacatgatttcaaattaagattggtacaaaagtgttctattttattcaattaaaccctacactatacaccttatgtcaacattagttgatgctacgacaaagcaatgaacattttatggattagttgatgctacaacaaagtatattgttagacttgctagattctatctactgttagcaccaaactaaccacaaaataacaatccctataactataggtggaggtgatgtaaaattaagggaggggcaattgtatatggtagtaccatagatatcaaaccttctcaacttagagctatgtctacatagttctagtcctatgaagcatcaaccattttcttatactaaactaaatatttttagaggaaaggggtggatccttactttcatctccatgagcttccaccccttcctatgagcttatccatcggttgaaagtttctcattgctagagcttactcgttattatcccatgccatctgacttttgtatatgtactattatctttgaagtcgtaggcatgtgtaaattcccacctcaagagtcaagatccattaatcctccaacacacccttaagacccaaaccataacacctaaatccaatttcaacatattttaggtgacatgggtatatgtgatattagttacttaatctagcaagctctattaatgatttttagtcagaaaatggttaatatgtttttagtggttgtactataattgaagaggcacatagagcaagtttttagtcgttgtattctaaacaatgattgatgtgtataaatttaataaattcattgttgcatcttgtgtttcatacatttgaaatgctttgtgcctaatctatatggatgaagaagtaaatccttctaaacttttccttccctgcaatctttttaaacacactctaaaccccaaatatctaatcctaacctctaaacctgatttaaattttctaatctagtccatttgtagtgcttttatatttagtccatttgccttatgtgcctcttgtgtataaatagcgtagagttctgtataatagtcaacaagttttgccttttgttgtcggatccattttcaatccttttgtctagttcacctattgttgttgtgaaaaaaatgtcacacattttttacttccccctataccacatactccatcacggactaatgatcttcaaggtatgtatgctcagtttaaatccatgtctccacatactccatcttaagttcaagtctctactttaaggtatgtaattttaaaactttgacgtattgtaattctataaggagcaaatctgaaaattaaataaggaaaaactggtaaaggcatgtttggaaatcggaacgcagacattttgttgttcctatgtttttctttaaataaactcattcgtgtaaaatttcttcaaaattcctctccttcgaacagatccttttgcccccggacccctttcctacgcttgcccaaacccacaaaaccctcgccgtcgcgccgcgcgattgcctctccggccgccgcgagcccgcgacactagtaacggtctacaccaccagaatgactgaagaattgaattccagcaaattcaagcttttgttttagccaagatttgagattcgatttgaagtgtggaagtccttccaatttgccaatcctatatttgatctctgctgtgctgcgttaaatccctaaacttcacagcgcggcgccggcccagccacgccggaagaggtcgccgcgtgaggtcagtgtccccgttgctgccgcctctaacccgaagcctaggccgctgccggtgcataacaaggagaatcaggcggaggggaaagtagcagaggagggggcagcaactgaggagggggagaagtaccgggcggaaccggaaatcttgccgctgccgccggccatggcgaagctggaagcttttgttttagccaagatttgagattcgatttgaagtgtggaagtccttccaatttgccaatcctatatttgatctctgctgtgctgcgttaaatccctaaacttcacagcgcggcgccggcccagccacgccggaagaggtcgccgcgtgaggtcagtgtccccgttgctgccgcctctaacccgaagcctaggccgctgccggtgcataacaaggagaatcaggcggaggggaaagtagcagaggagggggcagcaactgaggagggggagaagtaccgggcggaaccggaaatcttgccgctgccgccggccatggcgaagctgggcccggggcaggggctcgggtgcgaggcggcggaggggtcgctcgtgcccagccggaagcgggagtaccaagccctgcggcaagcacactgaggggaagcgcccgctatatgctatcgggttcaacttcatggacgcgcgctactacgacgtcttcgccaccgtcggcggcaaccgcgtaagccatcgactgctctctcctgtcgtcctttttttgtttctactgaggtttggggagttcttgttgattaatggcaaggtaaaactacgttgtttttttttgtgattttggtggtcggttttaggaagcggtcgcttttgattcaaatttgatctaaagctgaggcattcggttgtttttattggggacttgaggtctgtaatgttccgactattgtgatttgttttgccgaaacatggagtttgctagttcatttgatgaaaagctgcaacctttgacaaagaatttgtatcacttgggaaagtatagtgaggtgtggggaatcagatagtaccaatattactttgactatgattataagataatcttttaatgtcctttgtaacgaccatgctgcttttcgcttatcttgcctattgatcttgcaggtgacaacttaccgctgccttgagaatggtagtttcgctcttctacaagcttacgttgatgaggatgtaagaaagacaatgctcaatgacaatgcttttgcttgctgatttaatattgataatattctttctctaattcttgtgacgcctatttacctcagaaggatgagtcgttctatactctaagctgggctcgtgaccatgttgatggctcaccactgctggtggcagcaggaagcaatgggatcattcgggtcatcaattgtgctacagaaaagttagctaaggtaatctacccttatatttgtatgtgttcctatggtaaacttgaatgaagccttatttgcataattcaatatttcagttgtttatttgacatatatcactttatttatgatatctgatccagaaggtcttttggatttgctttagttaaggaatggtgcttgctacgcattaataccataagcaaactgtaccttttgctcacagaatattgttaattttgactacttcagtatgtccgttgtagtaaaaacaaatcaacttggtgtatctattttttccttgcttatacatagccaggagattgggcatgtggcatgtcaataaatactatcctataccatttgataggacacgcactgtgtcttatttggtagctctgtttacgtgattctgcagagctttgttggccatggcgactcaataaatgtgataagaactcaaccgttgaagccttcgctcatcatttctgcaagcaaggttatgcgatagtctgttcttaggttcatgtacctttttatttttataatctttctgaattttgacaccatttcatatggcattatctaataggatgaatctgttaggctatggaatgtccatacagggatctgtatcttgatatttgctggagctggaggtcatcgcaatgaagtattgagtgttgtaagtagtgcctgctattatgacattgtgcccttcaaaaaaaacattattatgacattatttttagaacattactaggttaaggtgcctttaatatggcgcactctttcagctcctgatattaccatttgttattgagcgttacatcagagataaaataaggctacctaatgactgctactgcttttgtactttgattacattagtcataaatgtactgatgaatacattattttgtcttaaggacttccatcctagtgatattgaacgttttgcaagttgtggcatggacaacactgtgaaaatctggtcaatgaaaggttagaaagctacttcaaagttgcttcatatttgcatgttgcgtgtcattgagttcaccaatgttgtcgcagaattttggctatatgttgacaaatcatattcatggactgaccttcatcaaagttccacaaaatatggccagtttccagtatgtttcacaatgcctatatccaattatcctggcaaggtcctgttggtgtctaatcctcatgccatcagactgacctgtttctttttgtttcaggtcttgattgctgcagtacactctaactatgttgattgaacaagatggcttggtgacttcatcctatcaaaggtgaaatttctgattcgtttaaatggatacaaatttctgtagcacggttgtcactcttttgtgggtttgacatgccactgtcttggttcatctattgctgtaccgtgcaagtgttcagttttttcaatcttttttctcagtgcttaatgaggggagattctatttgcagagtgttgtcaatgaaattgtgctttgggaaccgaagacaaaagaacagagtcctggggaggtaattcagtttaactttcccagaattgtattcctattataatgccatatatttacgcacagttgtaaactatttccagatccttagatttcaaggtactggctgccaatattaaatatgttccactgaagtaatatgattttctgttgcctcatagggaagcatcgatatccttcagaagtatcctgtcccagaatgtgacatttggtttatcaaattttcatgtgattttcacttcaatcagttggcgataggtaatatctctcatcaggattgtttctggtagaagttttatttaagattttttttgctctgtaaaatttcacacacgcacacatgcacccccacacacacacacatgcacgcacacccccacccacctgcacgcgcgcgtacacacacaccgcacacatatatatgactttttttcccacacaaatatttgctgtgtgagatatcagcaaataaattcgtatgtttgattatattcagagatataggaaaattgagtgctctaataccccatccactacttcaaacaggcaaccgtgaaggcaaaatctacgtgtggaaaaatacagtccagccctcctgtcctcattgctcggtagttttcactggaagagtttcagttattcttgtctcccacttgtatcgtcgcatgcttctggatgccaatgcttcatcattttcaggctgtataatcagcagtgtaaatcgccgataagacaaactgcagtgtccttcgatggaaggtacctcactctaatccatgctcaatttggtgtactgtctattctagcacttgcttttttcttggttctgcttgagaaattctcgattgcatgtcatatgctggtgcattttcttttttctgtttccgtggcggattggtaaaatgcgacgatgccttccttatctagcacaatccttggagctggtgaagacggcaccatctggcggtgggatgaagtggaccatccgagctccagaaactgaagaagtgttgccgctcaatgctggactgatggttacgctcggttggggttgtgatggttgaatccgttggcggaaagtgccacctggtgtttttttctagtcaaaatggttgatgttaacagaatattgaatgcttcgaatgttgaaagttgggatgcttgtgctggtactctgctccgcggacgagtgaacttagtttgttgcaactttgggaaccgttgtcatctgtttgttctgcatttctaaaaagagagcaaatttcaggatacatgttcttttttttcagtacaggaaaactaaggttgaggtattgctttgcaatttactctctctctctctctctcttaaaaaaactggatcttgcttcaacgatgcattccttgggtcatcggttttacttttgaaatcttgatagctgggcctaaagttaccaagcccactagtatcagaagtaataatatgatggctcctcccctgccttactgtcacgtgtaaactttcgaaactagcaggactgtagcatttagcgagctggttgtttgggttagagctcagcgtcgcaacttatggtaccgaggtcagtgtcaagatctatggcaccatggttcaatcacagttttagtcccaccaaaaatataaaggtgaagtttcgacaaaaaatggctagaataaaaaaaaacaggtccacatactgaggagaacacatgacagattcaccaaggattttgaattgaaagaggctaatgattgacaggatttgatcttcaattccacctcccgttgtcctgcttctactctaaagttcaagcgtggctcagtttggctatctgttataatttcaagaaatcctgatttctgttagcagtttactaggctattaggaggagctgggacaaaagaaaaacgagaattgacgaggacaaattcgcaattagttgggaaattgggggcacaattttcaatgcccacaaaattcactccccctacntntgcggnggaatggggtcanncctcantgtcccctgttnccgggacaagtntaactaacacatttccnnattnntn